(19) United States
(12) Patent Application Publication
(10) Pub. No.: US 2025/0102524 A1
(43) Pub. Date: Mar. 27, 2025

(54) HEMOGLOBIN G-MAKASSAR BINDING POLYPEPTIDES AND ANTIBODIES AND METHODS OF USING THE SAME

(71) Applicant: Beam Therapeutics Inc., Cambridge, MA (US)

(72) Inventors: Scott Haihua Chu, Cambridge, MA (US); Lisa Schlehuber, Cambridge, MA (US); Manuel Ortega, Cambridge, MA (US)

(21) Appl. No.: 18/908,540

(22) Filed: Oct. 7, 2024

Related U.S. Application Data
(63) Continuation of application No. PCT/US23/17876, filed on Apr. 7, 2023.
(60) Provisional application No. 63/329,109, filed on Apr. 8, 2022.

(51) Int. Cl. G01N 33/72; C07K 16/18

(52) U.S. Cl. CPC G01N 33/721; C07K 16/18; C07K 2317/33; G01N 2800/22

(57) ABSTRACT

Described and featured herein are binding polypeptides and antibodies, and antigen binding portions thereof, that specifically bind to the HbG-Makassar variant polypeptide or peptide and methods of using such binding polypeptides and antibodies to specifically bind, detect, identify, select, and/or isolate the HbG-Makassar variant polypeptide or peptide, for example, in a biological sample.

### CROSS REFERENCE TO RELATED APPLICATIONS

This application is a continuation of International Application No. PCT/US2023/017876, filed Apr. 7, 2023, which claims priority to and benefit of U.S. Provisional Application No. 63/329,109, filed Apr. 8, 2022, the entire contents of which are incorporated by reference herein.

### BACKGROUND

Sickle cell disease (SCD) is a condition that primarily affects people of sub-Saharan Africa and parts of the Mediterranean region, the Middle East and the Indian subcontinent; it affects more than 4.4 million people globally. SCD is caused by a single point mutation in the hemoglobin B (HBB) gene, which encodes the beta (β)-globin chain of hemoglobin A (HbA), the form of hemoglobin that is most prevalent in adults. Hemoglobin A transports oxygen as a tetramer of two alpha (α)-globin and two β-globin chains in red blood cells (erythrocytes). The mutant protein resulting from a point mutation causing SCD is termed Hemoglobin S, (HbS or HbSS), which polymerizes when deoxygenated and forms rigid aggregates that result in the characteristic sickle shape of the affected cells.

Sickled red blood cells have a shorter lifespan than normal cells, adhere to the walls of blood capillaries, and block blood flow to organs, causing painful vaso-occlusive crises (VOCs), ischemia, acute chest syndrome (ACS), bone or joint necrosis, renal failure, as well as a profound anemia associated with rapid splenic enlargement. Because cell sickling associated with SCD is difficult to eliminate entirely, a goal for therapeutics is to maintain the sickling below a certain threshold while boosting healthy red blood cell activity.

The hemoglobin (Hb) variant polypeptide, HbG-Makassar, is a naturally occurring β-globin polypeptide variant that is not associated with human disease. Certain therapeutic treatments for SCD under development and/or in the clinic aim to engineer a SCD patient's own cells to correct the mutation in the HBB gene that causes HbS to generate a normal hemoglobin product, or to produce a non-mutant form of hemoglobin, such as HbG-Makassar, thereby abrogating and/or managing the disease. Needed in the art are specific reagents and products that can be used to detect and identify with specificity and accuracy the HbG-Makassar polypeptide and that have minimal or no cross-reactivity with other forms of hemoglobin. Reagents and products, such as binding molecules and antibodies, that react specifically with the HbG-Makassar polypeptide are needed for both research and clinical utilities. The antibodies provided and described herein may satisfy these needs.

### SUMMARY

Featured herein are binding polypeptides, such as antibodies, or antigen binding portions thereof, that specifically bind to the HbG-Makassar hemoglobin (Hb) polypeptide, or a peptide thereof, and methods of using such binding polypeptides and antibodies for specifically binding to and identifying an HbG-Makassar polypeptide, e.g., in a biological sample.

Provided in one aspect is a binding polypeptide or an antigen binding portion thereof that specifically binds to an hemoglobin G (HbG) Makassar variant polypeptide, or a peptide thereof, but fails to detectably bind or binds at reduced levels to a wild-type beta (β)-globin polypeptide (or a peptide thereof) and/or sickle cell globin (HbS) polypeptide (or a peptide thereof). In an embodiment, the binding polypeptide of the above-delineated aspect comprises one or more complementarity determining regions (CDRs) which comprise or consist of heavy chain variable region (VH) CDRs and/or light chain variable region (VL) CDRs selected from the following: A) VH CDR1: GIDFSRYW; VH CDR2: INIDSSTI; VH CDR3: ARAYDGYSLDY; VL CDR1: SSVSY; VL CDR2: DTS; VL CDR3: RQWSSYPLT; B) VH CDR1: GYTFTNYF; VH CDR2: INPKNGGI; VH CDR3: ARGSANWGAY; VL CDR1: QRTNC; VL CDR2: HDL; VL CDR3: QQWSSYPLT; or C) VH CDR1: GYTFTSDW; VH CDR2: IYPRSGST; VH CDR3: ARGTYYGSRSYYFDY; VL CDR1: SSVSY; VL CDR2: DTS: VL CDR3: RQWSSYPLT,

Provided in another aspect is an anti-HbG-Makassar antibody, wherein the antibody or an antigen binding portion thereof specifically binds to an HbG-Makassar variant polypeptide, or a peptide thereof, and fails to detectably bind or binds at reduced levels to a beta (β)-globin polypeptide (or a peptide thereof) and/or sickle globin (HbS) polypeptide (or a peptide thereof). In an embodiment, the binding polypeptide or the antibody, or an antigen binding portion thereof, of the above-delineated aspect comprises or consists of VL CDR1: SSVSY, VL CDR2: DTS, and VL CDR3: RQWSSYPLT and VH CDR1: GIDFSRYW; VH CDR2: INIDSSTI; VH CDR3: ARAYDGYSLDY or VH CDR1: GYTFTSDW; VH CDR2: IYPRSGST; VH CDR3: ARGTYYGSRSYYFDY. In an embodiment, the binding polypeptide or the antibody, or an antigen binding portion thereof, comprises or consists of VL CDR1: QRTNC; VL CDR2: HDL; VL CDR3: QQWSSYPLT and VH CDR1: GYTFTNYF; VH CDR2: INPKNGGI; VH CDR3: ARGSANWGAY.

In an embodiment of the above-delineated aspects and embodiments thereof, the binding polypeptide or the antibody comprises:
a variable heavy chain (VH) domain comprising a CDR1 comprising amino acid sequence GIDFSRYW, a CDR2 comprising amino acid sequence INIDSSTI, and a CDR3 comprising amino acid sequence ARAYDGYSLDY; or
a variable heavy chain (VH) domain comprising a CDR1 comprising amino acid sequence GYTFTSDW, a CDR2 comprising amino acid sequence IYPRSGST, and a CDR3 comprising amino acid sequence ARGTYYGSRSYYFDY; and/or
a variable light chain (VL) domain comprising a CDR1 comprising amino acid sequence SSVSY, a CDR2 comprising amino acid sequence DTS, and a CDR3 comprising amino acid sequence RQWSSYPLT.

In an embodiment of the above-delineated aspects and embodiments thereof, the binding polypeptide or the antibody comprises a heavy chain variable domain (VH) sequence having at least 85% amino acid sequence identity to the amino acid sequence:

EVQLQESGGGLVQPGGSLKLSCAASGIDFSRYWMSWVRRAPGKGLEWIGE
INIDSSTINYAPSLKDKFIISRDNAKNTLYLQMSKVRSEDTALYYCARAY
DGYSLDYWGQGTSVTVSS,

and/or comprises a light chain variable domain (VL) sequence having at least 85% amino acid sequence identity to the amino acid sequence:

QIVLTQSPAIMSASPGEKVTMTCSTSSSVSYMFWYQQKPGSSPRLLIYDT
SNLASGVPVRFSGSGSGTSYSLTISRMEAEDAATYYCROWSSYPLTFGAG
TKLELK.

In an embodiment of the above-delineated aspects and embodiments thereof, the binding polypeptide or the antibody comprises a heavy chain variable domain (VH) sequence having at least 90% amino acid sequence identity to the amino acid sequence:

EVQLQESGGGLVQPGGSLKLSCAASGIDFSRYWMSWVRRAPGKGLEWIGE
INIDSSTINYAPSLKDKFIISRDNAKNTLYLQMSKVRSEDTALYYCARAY
DGYSLDYWGQGTSVTVSS,

and/or comprises a light chain variable domain (VL) sequence having at least 90% amino acid sequence identity to the amino acid sequence:

QIVLTQSPAIMSASPGEKVTMTCSTSSSVSYMFWYQQKPGSSPRLLIYDT
SNLASGVPVRFSGSGSGTSYSLTISRMEAEDAATYYCRQWSSYPLTFGAG
TKLELK.

In an embodiment of the above-delineated aspects and embodiments thereof, the binding polypeptide or the antibody comprises a heavy chain variable domain (VH) sequence having at least 95% amino acid sequence identity to the amino acid sequence:

EVQLQESGGGLVQPGGSLKLSCAASGIDFSRYWMSWVRRAPGKGLEWIGE
INIDSSTINYAPSLKDKFIISRDNAKNTLYLQMSKVRSEDTALYYCARAY
DGYSLDYWGQGTSVTVSS,

and/or comprises a light chain variable domain (VL) sequence having at least 95% amino acid sequence identity to the amino acid sequence:

QIVLTQSPAIMSASPGEKVTMTCSTSSSVSYMFWYQQKPGSSPRLLIYDT
SNLASGVPVRFSGSGSGTSYSLTISRMEAEDAATYYCRQWSSYPLTFGAG
TKLELK.

In an embodiment of the above-delineated aspects and embodiments thereof, the binding polypeptide or the antibody comprises a heavy chain variable domain (VH) sequence comprising or consisting of the amino acid sequence:

EVQLQESGGGLVQPGGSLKLSCAASGIDFSRYWMSWVRRAPGKGLEWIGE
INIDSSTINYAPSLKDKFIISRDNAKNTLYLQMSKVRSEDTALYYCARAY
DGYSLDYWGQGTSVTVSS,

and/or comprises a light chain variable domain (VL) sequence comprising or consisting of the amino acid sequence:

QIVLTQSPAIMSASPGEKVTMTCSTSSSVSYMFWYQQKPGSSPRLLIYDT
SNLASGVPVRFSGSGSGTSYSLTISRMEAEDAATYYCRQWSSYPLTFGAG
TKLELK.

In an embodiment of the above-delineated aspects and embodiments thereof, the binding polypeptide or the antibody comprises a heavy chain variable domain (VH) sequence having at least 85% amino acid sequence identity to the amino acid sequence:

EVLLQQSGPELVKPGASVKISCKASGYTFTNYFMNWVKQSHGKSLEWIGD
INPKNGGISYNQKFKGKATLIVDKSSSTAYMELRSLTSEDSAVYYCARGS
ANWGAYWGQGTLVTVSA,

and/or comprises a light chain variable domain (VL) sequence having at least 85% amino acid sequence identity to the amino acid sequence:

WWEDGYSWCSISHFQLPANQCLSHTVQRTNCSRPVSSNHVCISRGEGHHD
LQCQLKFPVRFSGSGSGTSYSLTISRMEAEDAATYYCQQWSSYPLTFGAG
TKLELK.

In an embodiment of the above-delineated aspects and embodiments thereof, the binding polypeptide or the antibody comprises a heavy chain variable domain (VH) sequence having at least 90% amino acid sequence identity to the amino acid sequence:

EVLLQQSGPELVKPGASVKISCKASGYTFTNYFMNWVKQSHGKSLEW
IGDINPKNGGISYNQKFKGKATLIVDKSSSTAYMELRSLTSEDSAVY
YCARGSANWGAYWGQGTLVTVSA,

and/or comprises a light chain variable domain (VL) sequence having at least 90% amino acid sequence identity to the amino acid sequence:

WWEDGYSWCSISHFQLPANQCLSHTVQRTNCSRPVSSNHVCISRGEG
HHDLQCQLKFPVRFSGSGSGTSYSLTISRMEAEDAATYYCQQWSSYP
LTFGAGTKLELK.

In an embodiment of the above-delineated aspects and embodiments thereof, the binding polypeptide or the antibody comprises a heavy chain variable domain (VH) sequence having at least 95% amino acid sequence identity to the amino acid sequence:

EVLLQQSGPELVKPGASVKISCKASGYTFTNYFMNWVKQSHGKSLEW
IGDINPKNGGISYNQKFKGKATLIVDKSSSTAYMELRSLTSEDSAVY
YCARGSANWGAYWGQGTLVTVSA,

and/or comprises a light chain variable domain (VL) sequence having at least 95% amino acid sequence identity to the amino acid sequence:

WWEDGYSWCSISHFQLPANQCLSHTVQRTNCSRPVSSNHVCISRGEG
HHDLQCQLKFPVRFSGSGSGTSYSLTISRMEAEDAATYYCQQWSSYP
LTFGAGTKLELK.

In an embodiment of the above-delineated aspects and embodiments thereof, the binding polypeptide or the antibody comprises a heavy chain variable domain (VH) sequence comprising or consisting of the amino acid sequence:

EVLLQQSGPELVKPGASVKISCKASGYTFTNYFMNWVKQSHGKSLEW
IGDINPKNGGISYNQKFKGKATLIVDKSSSTAYMELRSLTSEDSAVY
YCARGSANWGAYWGQGTLVTVSA,

and/or comprises a light chain variable domain (VL) sequence comprising or consisting of the amino acid sequence:

WWEDGYSWCSISHFQLPANQCLSHTVQRTNCSRPVSSNHVCISRGEG
HHDLQCQLKFPVRFSGSGSGTSYSLTISRMEAEDAATYYCQQWSSYP
LTFGAGTKLELK.

In an embodiment of the above-delineated aspects and embodiments thereof, the binding polypeptide or the antibody comprises a heavy chain variable domain (VH) sequence having at least 85% amino acid sequence identity to the amino acid sequence:

QVQLQQPGAELVKPGASVKMSCKASGYTFTSDWITWVKQRPGQGLEW
IGDIYPRSGSTNYNEKFKSKATLTVDISSNTAYMQLSSLTSEDSAVF
YCARGTYYGSRSYYFDYWGQGTTLTVSS,

and/or comprises a light chain variable domain (VL) sequence having at least 85% amino acid sequence identity to the amino acid sequence:

QIVLTQSPAIMSASPGEKVTMTCSTSSSVSYMFWYQQKPGSSPRLLI
YDTSNLASGVPVRFSGSGSGTSYSLTISRMEAEDAATYYCRQWSSYP
LTFGAGTKLELK.

In an embodiment of the above-delineated aspects and embodiments thereof, the binding polypeptide or the antibody comprises a heavy chain variable domain (VH) sequence having at least 90% amino acid sequence identity to the amino acid sequence:

QVQLQQPGAELVKPGASVKMSCKASGYTFTSDWITWVKQRPGQGLEW
IGDIYPRSGSTNYNEKFKSKATLTVDISSNTAYMQLSSLTSEDSAVF
YCARGTYYGSRSYYFDYWGQGTTLTVSS,

and/or comprises a light chain variable domain (VL) sequence having at least 90% amino acid sequence identity to the amino acid sequence:

QIVLTQSPAIMSASPGEKVTMTCSTSSSVSYMFWYQQKPGSSPRLLI
YDTSNLASGVPVRFSGSGSGTSYSLTISRMEAEDAATYYCRQWSSYP
LTFGAGTKLELK.

In an embodiment of the above-delineated aspects and embodiments thereof, the binding polypeptide or the antibody comprises a heavy chain variable domain (VH) sequence having at least 95% amino acid sequence identity to the amino acid sequence:

QVQLQQPGAELVKPGASVKMSCKASGYTFTSDWITWVKQRPGQGLEW
IGDIYPRSGSTNYNEKFKSKATLTVDISSNTAYMQLSSLTSEDSAVF
YCARGTYYGSRSYYFDYWGQGTTLTVSS,

and/or comprises a light chain variable domain (VL) sequence having at least 95% amino acid sequence identity to the amino acid sequence:

QIVLTQSPAIMSASPGEKVTMTCSTSSSVSYMFWYQQKPGSSPRLLI
YDTSNLASGVPVRFSGSGSGTSYSLTISRMEAEDAATYYCRQWSSYP
LTFGAGTKLELK.

In an embodiment of the above-delineated aspects and embodiments thereof, the binding polypeptide or the antibody comprises a heavy chain variable domain (VH) sequence comprising or consisting of the amino acid sequence:

QVQLQQPGAELVKPGASVKMSCKASGYTFTSDWITWVKQRPGQGLEW
IGDIYPRSGSTNYNEKFKSKATLTVDISSNTAYMQLSSLTSEDSAVF
YCARGTYYGSRSYYFDYWGQGTTLTVSS,

and/or comprises a light chain variable domain (VL) sequence comprising or consisting of the amino acid sequence:

QIVLTQSPAIMSASPGEKVTMTCSTSSSVSYMFWYQQKPGSSPRLLI
YDTSNLASGVPVRFSGSGSGTSYSLTISRMEAEDAATYYCRQWSSYP
LTFGAGTKLELK.

In an embodiment of the above-delineated aspects and embodiments thereof, the binding polypeptide or the antibody comprises an affinity tag.

In an embodiment of the above-delineated aspects and embodiments thereof, the binding polypeptide or the antibody, or a binding region thereof, comprises a detectable amino acid sequence.

In an embodiment, the binding polypeptide, the anti-HbG-Makassar antibody, or an antigen binding portion thereof, of any one of the above-delineated aspects and embodiments thereof, specifically binds to a hemoglobin G (HbG) Makassar peptide comprising the amino acid sequence VHLTPAEKSAVTA. In an embodiment, the binding polypeptide, the anti-HbG-Makassar antibody, or an antigen binding portion thereof, specifically binds to a hemoglobin G (HbG) Makassar peptide comprising the amino acid sequence VHLTPAEKSAVTA, but fails to detectably bind or binds at reduced levels to a sickle cell HbS peptide comprising the amino acid sequence VHLTPVEKSAVTA and/or to a wildtype beta-globin peptide comprising the amino acid sequence VHLTPEEKSAVTA.

In another aspect, a method of identifying an HbG-Makassar variant polypeptide or peptide is provided, in which the method involves contacting a sample with the polypeptide or the antibody of any of the above-delineated aspects and embodiments thereof for a time sufficient for the polypeptide or the antibody to bind to the HbG-Makassar variant polypeptide or peptide in the sample. In embodiments of the method, the sample comprises blood, plasma, serum, red blood cells, or a preparation obtained from bone marrow cells, cord blood-derived cells, or bone marrow-derived cells. In an embodiment, the sample is obtained from a patient. In an embodiment, the patient is undergoing testing for sickle cell disease (SCD). In an embodiment, the sample comprises cells obtained from a patient having sickle cell disease (SCD) and wherein the cells have been genetically edited to produce the HbG-Makassar polypeptide. In an embodiment, the cells are in vitro or ex vivo.

In another aspect, an isolated nucleic acid molecule that encodes the binding polypeptide or the antibody of any of the above-delineated aspects and embodiments thereof is provided. In an embodiment, the isolated nucleic acid molecule comprises a nucleic acid sequence having at least 85% sequence identity to the heavy chain variable domain (VH) nucleic acid sequence

gaggtgcagctgcaggagtctggaggtggcctggtgcagcctggagg
atccctgaaactctcctgtgcagcctcaggaatcgattttagtagat
actggatgagttgggttcggcgggctccagggaaaggactagaatgg
attggagaaattaatatagatagcagtacaataaactatgcaccatc
tctaaaggataaattcatcatctccagagacaacgccaaaaatacgc
tgtacctgcaaatgagcaaagtgagatctgaggacacagccctttat
tactgtgcaagggcctatgatggttattcgttggactactggggtca
aggaacctcagtcaccgtctcctcag

and/or comprises a nucleic acid sequence having at least 85% sequence identity to the light chain variable domain (VL) nucleic acid sequence

caaattgttctcacccagtctccagcaatcatgtctgcatctccagggg
agaaggtcaccatgacctgcagtaccagctcaagtgtaagttacatgtt
ctggtaccagcagaagccaggatcctcccccagactcctgatttatgac
acatccaacctggcttctggagtccctgttcgcttcagtggcagtgggt
ctgggacctcttactctctcacaatcagccgaatggaggctgaagatgc
tgccacttattactgccggcagtggagtagttaccccctcacgttcggt
gctgggaccaagctggagctgaaac.

In an embodiment, the isolated nucleic acid molecule comprises a nucleic acid sequence having at least 90% sequence identity to the heavy chain variable domain (VH) nucleic acid sequence

gaggtgcagctgcaggagtctggaggtggcctggtgcagcctggaggat
ccctgaaactctcctgtgcagcctcaggaatcgattttagtagatactg
gatgagttgggttcggcgggctccagggaaaggactagaatggattgga
gaaattaatatagatagcagtacaataaactatgcaccatctctaaagg
ataaattcatcatctccagagacaacgccaaaaatacgctgtacctgca
aatgagcaaagtgagatctgaggacacagccctttattactgtgcaagg
gcctatgatggttattcgttggactactggggtcaaggaacctcagtca
ccgtctcctcag

and/or comprises a nucleic acid sequence having at least 90% sequence identity to the light chain variable domain (VL) nucleic acid sequence

caaattgttctcacccagtctccagcaatcatgtctgcatctccagggg
agaaggtcaccatgacctgcagtaccagctcaagtgtaagttacatgtt
ctggtaccagcagaagccaggatcctcccccagactcctgatttatgac
acatccaacctggcttctggagtccctgttcgcttcagtggcagtgggt
ctgggacctcttactctctcacaatcagccgaatggaggctgaagatgc
tgccacttattactgccggcagtggagtagttaccccctcacgttcggt
gctgggaccaagctggagctgaaac.

In an embodiment, the isolated nucleic acid molecule comprises a nucleic acid sequence having at least 95% sequence identity to the heavy chain variable domain (VH) nucleic acid sequence

gaggtgcagctgcaggagtctggaggtggcctggtgcagcctggaggat
ccctgaaactctcctgtgcagcctcaggaatcgattttagtagatactg
gatgagttgggttcgggggctccagggaaaggactagaatggattggag
aaattaatatagatagcagtacaataaactatgcaccatctctaaagga
taaattcatcatctccagagacaacgccaaaaatacgctgtacctgcaa
atgagcaaagtgagatctgaggacacagccctttattactgtgcaaggg
cctatgatggttattcgttggactactggggtcaaggaacctcagtcac
cgtctcctcag

and/or comprises a nucleic acid sequence having at least 95% sequence identity to the light chain variable domain (VL) nucleic acid sequence

caaattgttctcacccagtctccagcaatcatgtctgcatctccagggg
agaaggtcaccatgacctgcagtaccagctcaagtgtaagttacatgtt
ctggtaccagcagaagccaggatcctcccccagactcctgatttatgac
acatccaacctggcttctggagtccctgttcgcttcagtggcagtgggt
ctgggacctcttactctctcacaatcagccgaatggaggctgaagatgc
tgccacttattactgccggcagtggagtagttaccccctcacgttcggt
gctgggaccaagctggagctgaaac.

In an embodiment, the isolated nucleic acid molecule comprises or consists of a heavy chain variable domain (VH) nucleic acid sequence

gaggtgcagctgcaggagtctggaggtggcctggtgcagcctggaggat
ccctgaaactctcctgtgcagcctcaggaatcgattttagtagatactg
gatgagttgggttcggcgggctccagggaaaggactagaatggattgga
gaaattaatatagatagcagtacaataaactatgcaccatctctaaagg
ataaattcatcatctccagagacaacgccaaaaatacgctgtacctgca
aatgagcaaagtgagatctgaggacacagccctttattactgtgcaagg
gcctatgatggttattcgttggactactggggtcaaggaacctcagtca
ccgtctcctcag

and/or comprises or consists of a light chain variable domain (VL) nucleic acid sequence

caaattgttctcacccagtctccagcaatcatgtctgcatctccagggg
agaaggtcaccatgacctgcagtaccagctcaagtgtaagttacatgtt
ctggtaccagcagaagccaggatcctcccccagactcctgatttatgac
acatccaacctggcttctggagtccctgttcgcttcagtggcagtgggt
ctgggacctcttactctctcacaatcagccgaatggaggctgaagatgc
tgccacttattactgccggcagtggagtagttaccccctcacgttcggt
gctgggaccaagctggagctgaaac.

In an embodiment, the isolated nucleic acid molecule comprises a nucleic acid sequence having at least 85% sequence identity to the heavy chain variable domain (VH) nucleic acid sequence

gaggtcctgctgcaacaatctggacctgagctggtgaagcctggggctt
cagtgaagatttcctgtaaggcttctggatacacgttcactaactactt
catgaactgggtgaagcagagccatggaaagagccttgagtggattgga
gatattaatcctaagaatggtggtattagttacaaccagaaatttaagg
gcaaggccacattgattgtagacaagtcctccagcacagcctacatgga
gctccgcagcctgacttctgaggactctgcagtctattattgtgcaaga
gggtcagctaactggggggcttactggggccaagggactctggtcactg
tctctgcag

and/or comprises a nucleic acid sequence having at least 85% sequence identity to the light chain variable domain (VL) nucleic acid sequence

tggtgggaagatggatacagttggtgcagcatcagccattttcagcttc
ctgctaatcagtgcctcagtcatactgtccagaggacaaattgttctcg
cccagtctccagcaatcatgtctgcatctccaggggagaaggtcaccat
gacctgcagtgccagctcaagttccctgttcgcttcagtggcagtgggt
ctgggacctcttactctctcacaatcagccgaatggaggctgaagatgc
tgccacttattactgccagcagtggagtagttaccccctcacgttcggt
gctgggaccaagctggaactgaaac.

In an embodiment, the isolated nucleic acid molecule comprises a nucleic acid sequence having at least 90% sequence identity to the heavy chain variable domain (VH) nucleic acid sequence

gaggtcctgctgcaacaatctggacctgagctggtgaagcctggggctt
cagtgaagatttcctgtaaggcttctggatacacgttcactaactactt
catgaactgggtgaagcagagccatggaaagagccttgagtggattgga
gatattaatcctaagaatggtggtattagttacaaccagaaatttaagg
gcaaggccacattgattgtagacaagtcctccagcacagcctacatgga
gctccgcagcctgacttctgaggactctgcagtctattattgtgcaaga
gggtcagctaactggggggcttactggggccaagggactctggtcactg
tctctgcag

and/or comprises a nucleic acid sequence having at least 90% sequence identity to the light chain variable domain (VL) nucleic acid sequence

tggtgggaagatggatacagttggtgcagcatcagccattttcagcttc
ctgctaatcagtgcctcagtcatactgtccagaggacaaattgttctcg
cccagtctccagcaatcatgtctgcatctccaggggagaaggtcaccat
gacctgcagtgccagctcaagttccctgttcgcttcagtggcagtgggt
ctgggacctcttactctctcacaatcagccgaatggaggctgaagatgc
tgccacttattactgccagcagtggagtagttaccccctcacgttcggt
gctgggaccaagctggaactgaaac.

In an embodiment, the isolated nucleic acid molecule comprises a nucleic acid sequence having at least 95% sequence identity to the heavy chain variable domain (VH) nucleic acid sequence

gaggtcctgctgcaacaatctggacctgagctggtgaagcctggggctt
cagtgaagatttcctgtaaggcttctggatacacgttcactaactactt
catgaactgggtgaagcagagccatggaaagagccttgagtggattgga
gatattaatcctaagaatggtggtattagttacaaccagaaatttaagg
gcaaggccacattgattgtagacaagtcctccagcacagcctacatgga
gctccgcagcctgacttctgaggactctgcagtctattattgtgcaaga
gggtcagctaactggggggcttactggggccaagggactctggtcactg
tctctgcag

and/or comprises a nucleic acid sequence having at least 95% sequence identity to the light chain variable domain (VL) nucleic acid sequence

tggtgggaagatggatacagttggtgcagcatcagccattttcagcttc
ctgctaatcagtgcctcagtcatactgtccagaggacaaattgttctcg
cccagtctccagcaatcatgtctgcatctccaggggagaaggtcaccat
gacctgcagtgccagctcaagttccctgttcgcttcagtggcagtgggt
ctgggacctcttactctctcacaatcagccgaatggaggctgaagatgc
tgccacttattactgccagcagtggagtagttaccccctcacgttcggt
gctgggaccaagctggaactgaaac.

In an embodiment, the isolated nucleic acid molecule comprises or consists of a heavy chain variable domain (VH) nucleic acid sequence

gaggtcctgctgcaacaatctggacctgagctggtgaagcctggggctt
cagtgaagatttcctgtaaggcttctggatacacgttcactaactactt
catgaactgggtgaagcagagccatggaaagagccttgagtggattgga
gatattaatcctaagaatggtggtattagttacaaccagaaatttaagg
gcaaggccacattgattgtagacaagtcctccagcacagcctacatgga
gctccgcagcctgacttctgaggactctgcagtctattattgtgcaaga
gggtcagctaactggggggcttactggggccaagggactctggtcactg
tctctgcag

and/or comprises or consists of a light chain variable domain (VL) nucleic acid sequence

tggtgggaagatggatacagttggtgcagcatcagccattttcagcttc
ctgctaatcagtgcctcagtcatactgtccagaggacaaattgttctcg
cccagtctccagcaatcatgtctgcatctccaggggagaaggtcaccat
gacctgcagtgccagctcaagttccctgttcgcttcagtggcagtgggt
ctgggacctcttactctctcacaatcagccgaatggaggctgaagatgc
tgccacttattactgccagcagtggagtagttaccccctcacgttcggt
gctgggaccaagctggaactgaaac.

In an embodiment, the isolated nucleic acid molecule comprises a nucleic acid sequence having at least 85% sequence identity to the heavy chain variable domain (VH) nucleic acid sequence

caggtccagctgcagcagcctggggctgagcttgtgaagcctggggc
ttcagtgaagatgtcctgcaaggcttctggctacaccttcaccagcg
actggataacctgggtgaagcagaggcctggacaaggccttgagtgg
attggagatatttatcctcgtagtggtagtactaactacaatgagaa
gttcaagagcaaggccacactgactgtagatatatcctccaacacag
cctacatgcagctcagcagcctgacatctgaggactctgcggtcttt
tactgtgcaagagggacttactacggtagtaggtcctactactttga
ctactggggccaaggcaccactctcacagtctcctcag

and/or comprises a nucleic acid sequence having at least 85% sequence identity to the light chain variable domain (VL) nucleic acid sequence

caaattgttctcacccagtctccagcaatcatgtctgcatctccagg
ggagaaggtcaccatgacctgcagtaccagctcaagtgtaagttaca
tgttctggtaccagcagaagccaggatcctcccccagactcctgatt
tatgacacatccaacctggcttctggagtccctgttcgcttcagtgg
cagtgggtctgggacctcttactctctcacaatcagccgaatggagg
ctgaagatgctgccacttattactgccggcagtggagtagttacccc
ctcacgttcggtgctgggaccaagctggagctgaaac.

In an embodiment, the isolated nucleic acid molecule comprises a nucleic acid sequence having at least 90% sequence identity to the heavy chain variable domain (VH) nucleic acid sequence

caggtccagctgcagcagcctggggctgagcttgtgaagcctggggc
ttcagtgaagatgtcctgcaaggcttctggctacaccttcaccagcg
actggataacctgggtgaagcagaggcctggacaaggccttgagtgg
attggagatatttatcctcgtagtggtagtactaactacaatgagaa
gttcaagagcaaggccacactgactgtagatatatcctccaacacag
cctacatgcagctcagcagcctgacatctgaggactctgcggtcttt
tactgtgcaagagggacttactacggtagtaggtcctactactttga
ctactggggccaaggcaccactctcacagtctcctcag

and/or comprises a nucleic acid sequence having at least 90% sequence identity to the light chain variable domain (VL) nucleic acid sequence

caaattgttctcacccagtctccagcaatcatgtctgcatctccagg
ggagaaggtcaccatgacctgcagtaccagctcaagtgtaagttaca
tgttctggtaccagcagaagccaggatcctcccccagactcctgatt
tatgacacatccaacctggcttctggagtccctgttcgcttcagtgg
cagtgggtctgggacctcttactctctcacaatcagccgaatggagg
ctgaagatgctgccacttattactgccggcagtggagtagttacccc
ctcacgttcggtgctgggaccaagctggagctgaaac.

In an embodiment, the isolated nucleic acid molecule comprises a nucleic acid sequence having at least 95% sequence identity to the heavy chain variable domain (VH) nucleic acid sequence

caggtccagctgcagcagcctggggctgagcttgtgaagcctggggc
ttcagtgaagatgtcctgcaaggcttctggctacaccttcaccagcg
actggataacctgggtgaagcagaggcctggacaaggccttgagtgg
attggagatatttatcctcgtagtggtagtactaactacaatgagaa
gttcaagagcaaggccacactgactgtagatatatcctccaacacag
cctacatgcagctcagcagcctgacatctgaggactctgcggtcttt
tactgtgcaagagggacttactacggtagtaggtcctactactttga
ctactggggccaaggcaccactctcacagtctcctcag

and/or comprises a nucleic acid sequence having at least 95% sequence identity to the light chain variable domain (VL) nucleic acid sequence

caaattgttctcacccagtctccagcaatcatgtctgcatctccagg
ggagaaggtcaccatgacctgcagtaccagctcaagtgtaagttaca
tgttctggtaccagcagaagccaggatcctcccccagactcctgatt
tatgacacatccaacctggcttctggagtccctgttcgcttcagtgg
cagtgggtctgggacctcttactctctcacaatcagccgaatggagg
ctgaagatgctgccacttattactgccggcagtggagtagttacccc
ctcacgttcggtgctgggaccaagctggagctgaaac.

In an embodiment, the isolated nucleic acid molecule comprises or consists of a heavy chain variable domain (VH) nucleic acid sequence

caggtccagctgcagcagcctggggctgagcttgtgaagcctggggc
ttcagtgaagatgtcctgcaaggcttctggctacaccttcaccagcg
actggataacctgggtgaagcagaggcctggacaaggccttgagtgg
attggagatatttatcctcgtagtggtagtactaactacaatgagaa
gttcaagagcaaggccacactgactgtagatatatcctccaacacag
cctacatgcagctcagcagcctgacatctgaggactctgcggtcttt
tactgtgcaagagggacttactacggtagtaggtcctactactttga
ctactggggccaaggcaccactctcacagtctcctcag

and/or comprises or consists of a light chain variable domain (VL) nucleic acid sequence

caaattgttctcacccagtctccagcaatcatgtctgcatctccagg
ggagaaggtcaccatgacctgcagtaccagctcaagtgtaagttaca
tgttctggtaccagcagaagccaggatcctcccccagactcctgatt
tatgacacatccaacctggcttctggagtccctgttcgcttcagtgg
cagtgggtctgggacctcttactctctcacaatcagccgaatggagg
ctgaagatgctgccacttattactgccggcagtggagtagttacccc
ctcacgttcggtgctgggaccaagctggagctgaaac.

In another aspect, a method of identifying and/or selecting a subject expressing an HbG-Makassar polypeptide is provided, in which the method involves (a) contacting a sample obtained from the subject with the binding polypeptide, the antibody, or an antigen binding portion thereof, of any one of the above-delineated aspects and embodiments thereof; (b) detecting specific binding between the binding polypeptide, the antibody, or the antigen binding portion thereof, and an HbG-Makassar polypeptide in the sample; and (c) identifying and/or selecting the subject as expressing an HbG-Makassar polypeptide based on the detecting step (b).

In another aspect, a method of monitoring a subject for the production of HbG-Makassar polypeptide is provided, in which the method involves: (a) contacting a sample obtained from the subject with the binding polypeptide, the antibody, or an antigen binding portion thereof, of any one of the above-delineated aspects and embodiments thereof, at a first time point and detecting specific binding between the binding polypeptide, the antibody, or the antigen binding portion thereof, and an HbG-Makassar polypeptide in the sample; (b) contacting a sample obtained from the subject with the binding polypeptide, the antibody, or the antigen binding portion thereof, of any one of the above-delineated aspects and embodiments thereof; at one or more additional time points and detecting specific binding between the binding polypeptide or the antibody and an HbG-Makassar polypeptide in the sample; and (c) monitoring that the subject is expressing the HbG-Makassar polypeptide by detecting the same level or a greater level of the HbG-Makassar polypeptide in the subject's sample in step (b) versus step (a).

In embodiments of the above-delineated methods, a relative or absolute level of at least 20%, at least 25%, at least 30%, at least 35%, or at least 40% of the hemoglobin in the sample from the subject (or in the subject or patient) is HbG-Makassar polypeptide in order to prevent sickling by the HbG-S in the subject or patient. In an embodiment of the above-delineated methods, the relative or absolute level of the HbG-Makassar polypeptide in the subject's sample is at least 30% of the hemoglobin in the sample to prevent sickling by HbG-S hemoglobin in the subject.

In another aspect, a method of assessing a relative or absolute level of HbG-Makassar hemoglobin in a subject expressing an HbG-Makassar polypeptide is provides, in which the method involves (a) contacting a sample obtained from the subject with the binding polypeptide, the antibody, or an antigen binding portion thereof, as set forth in any one of the above-delineated aspects and/or embodiments thereof; (b) detecting specific binding between the binding polypeptide, the antibody, or the antigen binding portion thereof, and an HbG-Makassar polypeptide in the sample; and (c) assessing a relative or absolute level of at least 30% of HbG-Makassar hemoglobin in the sample based on the detecting step (b); wherein said level of HbG-Makassar in the subject is sufficient to prevent sickling by HbG-S hemoglobin in the subject.

In embodiments of the above-delineated methods and/or embodiments thereof, the sample comprises cells or a cell preparation. In an embodiment, the cells are red blood cells (erythrocytes). In an embodiment, the cells have been genetically edited to express HbG-Makassar polypeptide prior to step (b) of the above-delineated method. In an embodiment, the cells are bone marrow-derived cells or cord blood-derived cells that have been genetically edited to express HbG-Makassar polypeptide. In an embodiment, the cell preparation comprises a lysate or supernatant. In an embodiment of the methods, the anti-HbG-Makassar antibody comprises 1C10.E3.G7, 1C10.C1.C7, or 5D6.F6.D2, or an antigen binding portion thereof. In an embodiment, the subject is a patient afflicted with sickle cell disease (SCD). In an embodiment, the subject is a patient afflicted with a hemoglobinopathy that is treatable by the expression and production of the HbG-Makassar polypeptide. In an embodiment, the cells are in vitro or ex vivo.

In another aspect, a composition comprising the binding polypeptide or the antibody of any one of the above-delineated aspects and embodiments thereof, or an antigen binding fragment thereof, is provided.

In an aspect, a composition comprising the isolated nucleic acid molecule of any one of the above-delineated aspects and embodiments thereof is provided.

In an aspect, a vector comprising a nucleic acid molecule that encodes the binding polypeptide or the antibody of any one of the above-delineated aspects and embodiments thereof is provided.

In an aspect, a vector comprising the isolated nucleic acid molecule of any one of the above-delineated aspects and embodiments thereof is provided.

In an embodiment, the vector of the above-delineated aspects is an expression vector.

In an embodiment, the expression vector is a viral or non-viral expression vector. In an embodiment, the vector of the above-delineated aspects, encodes an affinity tag or a detectable amino acid sequence operably linked to the binding polypeptide or to the antibody, or to an antigen binding portion thereof.

In an aspect, a cell comprising the vector of any one of the above-delineated aspects and embodiments thereof is provided.

In an aspect, a composition comprising the vector of any one of the above-delineated aspects and embodiments thereof is provided.

In an aspect, a composition comprising the cell of the above-delineated aspect is provided.

In an aspect, a kit comprising the binding polypeptide, the antibody, or an antigen binding portion thereof, of any one of the above-delineated aspects and embodiments thereof is provided.

In an aspect, a kit comprising the isolated nucleic acid molecule of any one of the above-delineated aspects and embodiments thereof is provided.

In an aspect, a kit comprising the composition of any one of the above-delineated aspects and embodiments thereof is provided.

In an aspect, a kit comprising the vector of any one of the above-delineated aspects and embodiments thereof, or the cell of the above-delineated aspect is provided.

In an embodiment, the kit of any of the above-delineated aspects and embodiments thereof, further comprises instructions for use in detecting or identifying the presence of HbG-Makassar polypeptide or peptide in a sample.

### Definitions

Unless defined otherwise, all technical and scientific terms used herein have the meaning commonly understood by a person skilled in the art to which the aspects and embodiments described herein belong. The following references provide one of skill with a general definition of many of the terms used in the described aspects and embodiments: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a small compound, protein, polypeptide, nucleic acid molecule or a fragment thereof. In various embodiments, the agent is an anti-Makassar antibody or antigen binding fragment thereof that binds an HbG-Makassar polypeptide.

By “ameliorate” is meant decrease, suppress, attenuate, diminish, arrest, abate, abrogate, or stabilize the development or progression of a disease. In an embodiment, the disease is Sickle cell disease (SCD), also called sickle cell anemia or hemoglobin S (HbS) disease.

By “affinity tag” or “protein tag,” is meant a peptide sequence that is attached to a protein. In embodiments, the peptide sequence is genetically fused to a protein (e.g., a recombinant protein, a binding polypeptide or an antibody. Such tags, which may be fused to either or both the N (amino)-terminus or the C (carboxy)-terminus, are typically removable by chemical or enzymatic agents, such as proteolysis or intein splicing. If the tag is inserted into the coding sequence of the protein of interest, it is termed an internal tag. In an embodiment, an affinity tag is fused or appended to a protein in order to purify or isolate the protein from a crude biological source or a mixture of components using an affinity technique. Nonlimiting examples of peptide/protein tags for this purpose include chitin binding protein (CBP), maltose binding protein (MBP), Strep or streptavidin tag, and glutathione-S-transferase (GST). Poly-histidine (Poly-His) tags, which typically comprise 5-10 histidine residues bound by a nickel or cobalt chelate, are commonly used, as they bind to metal matrices. Solubilization tags are used, especially for recombinant proteins expressed in chaperone-deficient species such as E. coli, to assist in proper protein folding and to prevent precipitation. Solubilization tags include thioredoxin (TRX) and poly(NANP). Some affinity tags have a dual role as a solubilization agent, such as MBP, and GST. Chromatography tags, e.g., polyanionic amino acids, such as FLAG-tag, are used to alter the chromatographic properties of a protein to provide different resolution for a particular separation technique. Epitope tags are short peptide sequences which are chosen because high-affinity antibodies can be reliably produced in many different species. Epitope tags are usually derived from viral genes and include ALFA-tag (helical peptide tag (e.g., SRLEEELRRRLTE) for biochemical and microscopy applications, and recognized by a repertoire of single-domain antibodies), V5-tag (a peptide tag (e.g., GKPIPNPLLGLDST) that is recognized by an anti-V5-tag antibody, Myc-tag (a peptide tag (e.g., EQKLISEEDL) derived from c-myc that is recognized by an anti-Myc tag antibody, HA-tag (a peptide tag (e.g., YPYDVPDYA) derived from hemagglutinin that is recognized by an anti-HA antibody, Spot-tag (a peptide recognized by a nanobody (e.g., PDRVRAVSHWSS) for immunoprecipitation, affinity purification, immunofluorescence), T7-tag (an epitope tag derived from the T7 major capsid protein of the T7 gene (e.g., MASMTGGQQMG), and NE-tag (an 18-amino-acid synthetic peptide (e.g., TKENPRSNQEESYDDNES) recognized by a monoclonal IgG1 antibody), which are useful in performing Western blots, immunofluorescence analyses, and immunoprecipitation analyses, as well as for antibody purification. Fluorescence tags, such as green fluorescent protein (GFP) and known variants of GFP, provide a visual readout, e.g., via fluorescence. Protein tags may allow specific enzymatic modification (such as biotinylation by biotin ligase) or chemical modification (such as reaction with FlAsH-EDT2, a pro-fluorescent, membrane-permeable biarsenical compound that binds covalently to a tetracysteine sequence (CCPGCC), for fluorescence imaging).

As used herein, the term “antibody” (Ab) refers to an immunoglobulin molecule that specifically binds to, or is immunologically reactive with, a particular antigen, and antigen binding fragments thereof. Exemplary antibodies encompass polyclonal, monoclonal, genetically and molecularly engineered and otherwise modified forms of antibodies, including, but not limited to, chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)2, Fab, Fv, rlgG, and scFv fragments. Antibodies (immunoglobulins) comprise two heavy chains linked together by disulfide bonds, and two light chains, with each light chain being linked to a respective heavy chain by disulfide bonds in a “Y” shaped configuration. Each heavy chain has at one end a variable domain (VH) followed by a number of constant domains (CH). Each light chain has a variable domain (VL) at one end and a constant domain (CL) at its other end. The variable domain of the light chain (VL) is aligned with the variable domain of the heavy chain (VL), and the light chain constant domain (CL) is aligned with the first constant domain of the heavy chain (CH1). The variable domains of each pair of light and heavy chains form the antigen binding site. The isotype of the heavy chain (gamma, alpha, delta, epsilon or mu) determines the immunoglobulin class (IgG, IgA, IgD, IgE or IgM, respectively). The light chain is either of two isotypes (kappa (κ) or lambda (λ)) found in all antibody classes. The terms “antibody” or “antibodies” include intact antibodies, such as polyclonal antibodies or monoclonal antibodies (mAbs), as well as proteolytic portions or fragments thereof, such as the Fab or F(ab′)2 fragments, that are capable of specifically binding to a target protein. Antibodies may include chimeric antibodies; recombinant and engineered antibodies, and antigen binding fragments thereof.

Exemplary functional antibody fragments comprising whole or essentially whole variable regions of both the light and heavy chains are defined as follows: (i) Fv, defined as a genetically engineered fragment consisting of the variable region of the light chain and the variable region of the heavy chain expressed as two chains; (ii) single-chain Fv (“scFv”), a genetically engineered single-chain molecule including the variable region of the light chain and the variable region of the heavy chain, linked by a suitable polypeptide linker; (iii) Fab, a fragment of an antibody molecule containing a monovalent antigen-binding portion of an antibody molecule, obtained by treating an intact antibody with the enzyme papain to yield the intact light chain and the Fd fragment of the heavy chain, which consists of the variable and CH1 domains thereof; (iv) Fab′, a fragment of an antibody molecule containing a monovalent antigen-binding portion of an antibody molecule, obtained by treating an intact antibody with the enzyme pepsin, followed by reduction (two Fab′ fragments are generated per antibody molecule); and (v) F(ab′)2, a fragment of an antibody molecule containing a monovalent antigen-binding portion of an antibody molecule, obtained by treating an intact antibody with the enzyme pepsin (i.e., a dimer of Fab′ fragments held together by two disulfide bonds). Exemplary anti-HbG-Makassar antibodies are described herein and are useful in the various aspects and embodiments described herein. Without intending to be limiting, the antibodies described herein provide the ability to recognize and bind specifically to the HbG-Makassar variant polypeptide or peptide (as protein antigen) or to epitopes within the HbG-Makassar variant polypeptide (antigen domains, regions, or determinants).

By “ß-globin (HBB) protein” is meant a polypeptide or fragment thereof having at least about 95% amino acid sequence identity to NCBI Accession No. NP_000509 (GenBank Ref. No. AAA52634.1)/UniProtKB Ref: P68871 (HBB_Human). In particular embodiments, a ß-globin protein comprises one or more alterations relative to the following reference sequence. In an embodiment, a human HbG Makassar variant ß-globin protein or peptide comprises an E6A mutation. In an embodiment, a human ß-globin protein or peptide associated with sickle cell disease comprises an E6V (also termed E7V) mutation. An exemplary ß-globin amino acid sequence is provided below.

| | |
|---|---|
| 1 | MVHLTPEEKS AVTALWGKVN VDEVGGEALG RLLVVYPWTQ RFFESFGDLS TPDAVMGNPK |
| 61 | VKAHGKKVLG AFSDGLAHLD NLKGTFATLS ELHCDKLHVD PENFRLLGNV LVCVLAHHFG |
| 121 | KEFTPPVQAA YQKVVAGVAN ALAHKYH |

By “HBB polynucleotide” is meant a nucleic acid molecule encoding ß-globin protein or fragment thereof. The sequence of an exemplary HBB polynucleotide, which is referenced at NCBI Accession No. NM 000518, is provided below:

| | |
|---|---|
| 1 | acatttgctt ctgacacaac tgtgttcact agcaacctca aacagacacc atggtgcatc |
| 61 | tgactcctga ggagaagtct gccgttactg ccctgtgggg caaggtgaac gtggatgaag |
| 121 | ttggtggtga ggccctgggc aggctgctgg tggtctaccc ttggacccag aggttctttg |
| 181 | agtcctttgg ggatctgtcc actcctgatg ctgttatggg caaccctaag gtgaaggctc |
| 241 | atggcaagaa agtgctcggt gcctttagtg atggcctggc tcacctggac aacctcaagg |
| 301 | gcacctttgc cacactgagt gagctgcact gtgacaagct gcacgtggat cctgagaact |
| 361 | tcaggctcct gggcaacgtg ctggtctgtg tgctggccca tcactttggc aaagaattca |
| 421 | ccccaccagt gcaggctgcc tatcagaaag tggtggctgg tgtggctaat gccctggccc |
| 481 | acaagtatca ctaagctcgc tttcttgctg tccaatttct attaaaggtt cctttgttcc |
| 541 | ctaagtccaa ctactaaact gggggatatt atgaagggcc ttgagcatct ggattctgcc |
| 601 | taataaaaaa catttatttt cattgcaa |

The terms “Makassar globin,” “HbG-Makassar,” “HbG,” or “Makassar” (hemoglobin) refer to a human beta (β)-hemoglobin (human β-globin) variant polypeptide having the mutation E→A at position 6, or a peptide thereof. The Makassar variant refers to an asymptomatic, naturally-occurring variant or mutation (E6A) of human β-globin. The terms “Makassar (“Mksr”) globin,” “HbG-Makassar,” “HbG,” or “Makassar” are used interchangeably herein to refer to this human ß-globin variant polypeptide or peptide. The Makassar mutation was first identified in Indonesia and was called HbG-Makassar. (Mohamad, A. S. et al., 2018, Hematol. Rep., 10(3):7210 (doi:10.4081/hr.2018.7210). The HbG-Makassar mobility is slower than that of normal (e.g., wild type) 0-hemoglobin when the variant polypeptide is subjected to electrophoresis. The HbG Makassar polypeptide has its anatomical abnormality at the β-6 or A3 location where the glutamyl residue typically is replaced by an alanyl residue. The substitution of a single amino acid in the gene encoding the β-globin subunit β-6 glutamyl to valine results in sickle cell disease. Routine procedures, such as isoelectric focusing, hemoglobin electrophoresis separation by cation-exchange High Performance Liquid Chromatography (HPLC) and cellulose acetate electrophoresis, have been unable to separate the HbG-Makassar and HbS globin (also termed HbG-S) forms, as they were found to have identical properties when analyzed by these methods. Consequently, HbG-Makassar and HbG-S (or HbS) have frequently been incorrectly identified and mistaken for each other by those skilled in the art, thereby leading to misdiagnosis of Sickle Cell Disease (SCD). In embodiments, a relative or absolute level of at least 20%, at least 25%, at least 30%, at least 35%, at least 40%, at least 45%, at least 50%, or greater of hemoglobin polypeptide in a subject or patient, e.g., a patient having SCD, (or detected in a sample from the subject or patient) is optimally HbG-Makassar hemoglobin in order to prevent sickling by HbG-S hemoglobin in the subject or patient with SCD. In an embodiment, a relative or absolute level of at least 30% of the hemoglobin in the subject or patient (or in a sample from the subject or patient) is HbG-Makassar polypeptide in order to prevent sickling by the HbG-S in the subject or patient. In an embodiment, a relative or absolute level of at least 25% of the hemoglobin in the subject or patient (or in a sample from the subject or patient) is HbG-Makassar polypeptide in order to prevent sickling by the HbG-S in the subject or patient. In an embodiment, a relative or absolute level of at least 30% of the hemoglobin in the subject or patient (or in a sample from the subject or patient) is HbG-Makassar polypeptide in order to prevent sickling by the HbG-S in the subject or patient. In an embodiment, a relative or absolute level of at least 35% of the hemoglobin in the subject or patient (or in a sample from the subject or patient) is HbG-Makassar polypeptide in order to prevent sickling by the HbG-S in the subject or patient.

By “anti-HbG-Makassar antibody 1C10.E3.G7” or “anti-Makassar 1C10.E3.G7 antibody” is meant an antibody having at least about 85% amino acid sequence identity to an antibody sequence of antibody 1C10.E3.G7 or comprising VH and/or VL CDRs1-3 of 1C10.E3.G7 or antigen binding fragments thereof, wherein each of the antibody, CDRs, and antigen binding fragments specifically bind to an HbG-Makassar polypeptide, but fail to detectably bind or have only reduced binding to a wild-type β-globin polypeptide or sickle cell globin (HbS or HbG-S) polypeptide. In embodiments, the antibody or antigen binding fragment thereof has at least 90%, 93%, 95%, 98%, 99% or 100% amino acid sequence identity to an antibody sequence of antibody 1C10.E3.G7. Exemplary variable region sequences for antibody 1C10.E3.G7 are provided below:

1C10.E3.G7 Heavy chain variable region (VH)

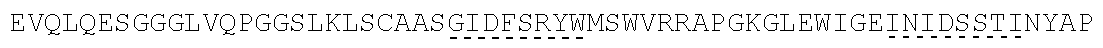

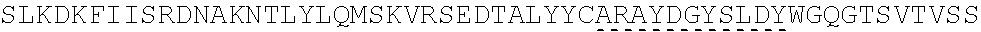

1C10.E3.G7 Light chain variable region (VL)

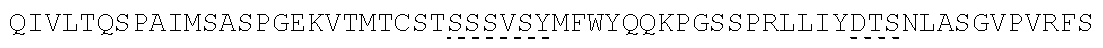

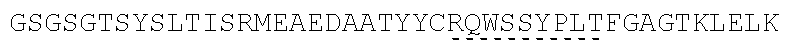

The three complementarity determining regions (CDRs), i.e., CDR1, CDR2 and CDR3, are underlined in the 1C10.E3.G7 antibody VH and VL region sequences shown supra. In particular, the three CDRs of the 1C10.E3.G7 antibody VH region are as follows:

VH CDR1:
GIDFSRYW
VH CDR2:
INIDSSTI
VH CDR3:
ARAYDGYSLDY

The three CDRs of the 1C10.E3.G7 antibody VL region are as follows:

VL CDR1:
SSVSY
VL CDR2:
DTS
VL CDR3:
RQWSSYPLT

The four framework (FR) regions, i.e., FR1, FR2, FR3, and FR4, of the 1C10.E3.G7 antibody are located on either side of each of the CDRs in VH and VL region sequences shown supra, In particular, the four FRs of the 1C10.E3.G7 antibody VH region are as follows:

VH FR1:
EVQLQESGGGLVQPGGSLKLSCAAS
VH FR2:
MSWVRRAPGKGLEWIGE
VH FR3:
NYAPSLKDKFIISRDNAKNTLYLQMSKVRSEDTALYYC
VH FR4:
WGQGTSVTVSS

The four FRs of the 1C10.E3.G7 antibody VL region are as follows:

VL FR1:
QIVLTQSPAIMSASPGEKVTMTCSTS
VL FR2:
MFWYQQKPGSSPRLLIY
VL FR3:
NLASGVPVRFSGSGSGTSYSLTISRMEAEDAATYYC
VL FR4:
FGAGTKLELK

By “1C10.E3.G7 polynucleotide” is meant a nucleic acid molecule (e.g., DNA) encoding at least a fragment of a 1C10.E3.G7 antibody. In an embodiment, the encoded fragment has antigen binding activity.

By “1C10.E3.G7 VH polynucleotide” is meant a nucleic acid molecule (e.g., DNA) encoding a variable region of the heavy chain of the 1C10.E3.G7 antibody. In embodiments, a polynucleotide having at least 80%, at least 85%, at least 90%, at least 93%, at least 95%, at least 98%, at least 99% or 100% amino acid sequence identity to the 1C10.E3.G7 VH polynucleotide sequence is encompassed.

1C10.E3.G7 VH polynucleotide sequence
gaggtgcagctgcaggagtctggaggtggcctggtgcagcctggagga
tccctgaaactctcctgtgcagcctcaggaatcgattttagtagatac
tggatgagttgggttcggcgggctccagggaaaggactagaatggatt
ggagaaattaatatagatagcagtacaataaactatgcaccatctcta
aaggataaattcatcatctccagagacaacgccaaaaatacgctgtac
ctgcaaatgagcaaagtgagatctgaggacacagccctttattactgt
gcaagggcctatgatggttattcgttggactactggggtcaaggaacc
tcagtcaccgtctcctcag

By “1C10.E3.G7 VL polynucleotide” is meant a nucleic acid molecule (e.g., DNA) encoding a variable region of the light chain of the 1C10.E3.G7 antibody. In embodiments, a polynucleotide having at least 80%, at least 85%, at least 90%, at least 93%, at least 95%, at least 98%, at least 99% or 100% amino acid sequence identity to the 1C10.E3.G7 VL polynucleotide sequence is encompassed.

1C10.E3.G7 VL polynucleotide sequence
caaattgttctcacccagtctccagcaatcatgtctgcatctccagggga
gaaggtcaccatgacctgcagtaccagctcaagtgtaagttacatgttct
ggtaccagcagaagccaggatcctcccccagactcctgatttatgacaca
tccaacctggcttctggagtccctgttcgcttcagtggcagtgggtctgg
gacctcttactctctcacaatcagccgaatggaggctgaagatgctgcca
cttattactgccggcagtggagtagttaccccctcacgttcggtgctggg
accaagctggagctgaaac.

By “anti-HbG-Makassar antibody 1C10.C1.C7” or “anti-Makassar 1C10.C1.C7 antibody” is meant an antibody having at least about 85% amino acid sequence identity to an antibody sequence of antibody 1C10.C1.C7 or comprising VH and/or VL CDRs1-3 of 1C10.C1.C7 or antigen binding fragments thereof, wherein each of the antibody, CDRs, and antigen binding fragments specifically bind to an HbG-Makassar polypeptide, but fail to detectably bind or have only reduced binding to a wild-type β-globin polypeptide or sickle cell globin (HbS or HbG-S) polypeptide. In embodiments, the antibody or antigen binding fragment thereof has at least 90%, 93%, 95%, 98%, 99% or 100% amino acid sequence identity to an antibody sequence of antibody 1C10.C1.C7. Exemplary variable region sequences for antibody 1C10.C1.C7 are provided below:

1C10.C1.C7 Heavy chain variable region (VH)

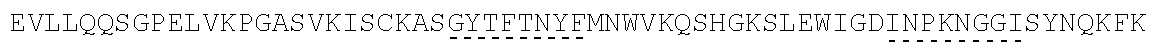

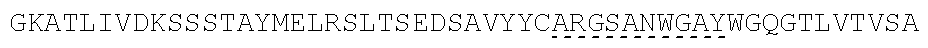

1C10.C1.C7 Light chain variable region (VL)

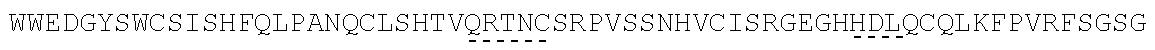

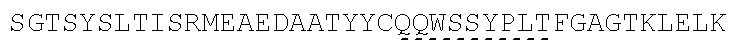

The three complementarity determining regions (CDRs), i.e., CDR1, CDR2 and CDR3, are underlined in the 1C10.C1.C7 antibody VH and VL region sequences shown supra. In particular, the three CDRs of the 1C10.C1.C7 antibody VH region are as follows:

VH CDR1:
GYTFTNYF
VH CDR2:
INPKNGGI
VH CDR3:
ARGSANWGAY

The three CDRs of the 1C10.C1.C7 antibody VL region are as follows: VL CDR1: QRTNC

VL CDR2:
HDL
VL CDR3:
QQWSSYPLT

The four framework (FR) regions, i.e., FR1, FR2, FR3, and FR4, of the 1C10.C1.C7 antibody are located on either side of each of the CDRs in VH and VL region sequences shown supra, In particular, the four FRs of the 1C10.C1.C7 antibody VH region are as follows:

VH FR1:
EVLLQQSGPELVKPGASVKISCKAS
VH FR2:
MNWVKQSHGKSLEWIGD
VH FR3:
SYNQKFKGKATLIVDKSSSTAYMELRSLTSEDSAVYYC
VH FR4:
WGQGTLVTVSA

The four FRs of the 1C10.C1.C7 antibody VL region are as follows:

VL FR1:
WWEDGYSWCSISHFQLPANQCLSHTV
VL FR2:
SRPVSSNHVCISRGEGH
VL FR3:
QCQLKFPVRFSGSGSGTSYSLTISRMEAEDAATYYC
VL FR4:
FGAGTKLELK

By “1C10.C1.C7 polynucleotide” is meant a nucleic acid molecule (e.g., DNA) encoding at least a fragment of a 1C10.C1.C7 antibody. In an embodiment, the encoded fragment has antigen binding activity.

By “1C10.C1.C7 VH polynucleotide” is meant a nucleic acid molecule (e.g., DNA) encoding a variable region of the heavy chain of the 1C10.C1.C7 antibody. In embodiments, a polynucleotide having at least 80%, at least 85%, at least 90%, at least 93%, at least 95%, at least 98%, at least 99% or 100% amino acid sequence identity to the 1C10.C1.C7 VH polynucleotide sequence is encompassed.

1C10.C1.C7 VH polynucleotide sequence:
gaggtcctgctgcaacaatctggacctgagctggtgaagcctggggcttc
agtgaagatttcctgtaaggcttctggatacacgttcactaactacttca
tgaactgggtgaagcagagccatggaaagagccttgagtggattggagat
attaatcctaagaatggtggtattagttacaaccagaaatttaagggcaa
ggccacattgattgtagacaagtcctccagcacagcctacatggagctcc
gcagcctgacttctgaggactctgcagtctattattgtgcaagagggtca
gctaactggggggcttactggggccaagggactctggtcactgtctctgc
ag.

By “1C10.C1.C7 VL polynucleotide” is meant a nucleic acid molecule (e.g., DNA) encoding a variable region of the light chain of the 1C10.C1.C7 antibody. In embodiments, a polynucleotide having at least 80%, at least 85%, at least 90%, at least 93%, at least 95%, at least 98%, at least 99% or 100% amino acid sequence identity to the 1C10.C1.C7 VL polynucleotide sequence is encompassed.

1C10.C1.C7 VL polynucleotide sequence:
tggtgggaagatggatacagttggtgcagcatcagccattttcagcttcc
tgctaatcagtgcctcagtcatactgtccagaggacaaattgttctcgcc
cagtctccagcaatcatgtctgcatctccaggggagaaggtcaccatgac
ctgcagtgccagctcaagttccctgttcgcttcagtggcagtgggtctgg
gacctcttactctctcacaatcagccgaatggaggctgaagatgctgcca
cttattactgccagcagtggagtagttaccccctcacgttcggtgctggg
accaagctggaactgaaac.

By “anti-HbG-Makassar antibody 5D6.F6.D2” or “anti-Makassar 5D6.F6.D2 antibody” is meant an antibody having at least about 85% amino acid sequence identity to an antibody sequence of antibody 5D6.F6.D2 or comprising VH and/or VL CDRs1-3 of 5D6.F6.D2 or antigen binding fragments thereof, wherein each of the antibody, CDRs, and antigen binding fragments specifically bind to an HbG-Makassar polypeptide, but fail to detectably bind or have only reduced binding to a wild-type β-globin polypeptide or sickle cell globin (HbS or HbG-S) polypeptide. In embodiments, the antibody or antigen binding fragment thereof has at least 90%, 93%, 95%, 98%, 99% or 100% amino acid sequence identity to an antibody sequence of antibody 5D6.F6.D2. Exemplary variable region sequences for antibody 5D6.F6.D2 are provided below: 5D6.F6.D2 Heavy chain variable region (VH)

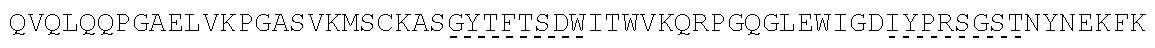

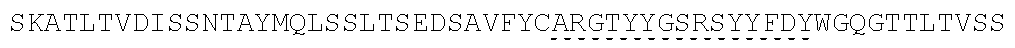

5D6.F6.D2 Light chain variable region (VL)

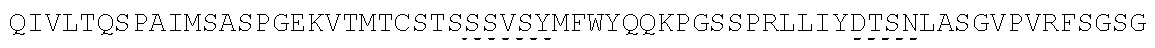

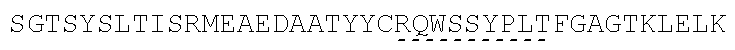

The three complementarity determining regions (CDRs), i.e., CDR1, CDR2 and CDR3, are underlined in the 5D6.F6.D2 antibody VH and VL region sequences shown supra. In particular, the three CDRs of the 5D6.F6.D2 antibody VH region are as follows:

VH CDR1:
GYTFTSDW
VH CDR2:
IYPRSGST
VH CDR3:
ARGTYYGSRSYYFDY

The three CDRs of the 5D6.F6.D2 antibody VL region are as follows:

VL CDR1:
SSVSY
VL CDR2:
DTS
VL CDR3:
RQWSSYPLT

The four framework (FR) regions, i.e., FR1, FR2, FR3, and FR4, of the 5D6.F6.D2 antibody are located on either side of each of the CDRs in VH and VL region sequences shown supra, In particular, the four FRs of the 5D6.F6.D2 antibody VH region are as follows:

VH FR1:
QVQLQQPGAELVKPGASVKMSCKAS
VH FR2:
ITWVKQRPGQGLEWIGD
VH FR3:
NYNEKFKSKATLTVDISSNTAYMQLSSLTSEDSAVFYC
VH FR4:
WGQGTTLTVSS

The four FRs of the 5D6.F6.D2 antibody VL region are as follows:

VL FR1:
QIVLTQSPAIMSASPGEKVTMTCSTS
VL FR2:
MFWYQQKPGSSPRLLIY
VL FR3:
NLASGVPVRFSGSGSGTSYSLTISRMEAEDAATYYC
VL FR4:
FGAGTKLELK

By “5D6.F6.D2 polynucleotide” is meant a nucleic acid molecule (e.g., DNA) encoding at least a fragment of a 5D6.F6.D2 antibody. In an embodiment, the encoded fragment has antigen binding activity.

By “5D6.F6.D2 VH polynucleotide” is meant a nucleic acid molecule (e.g., DNA) encoding a variable region of the heavy chain of the 5D6.F6.D2 antibody. In embodiments, a polynucleotide having at least 80%, at least 85%, at least 90%, at least 93%, at least 95%, at least 98%, at least 99% or 100% amino acid sequence identity to the 5D6.F6.D2 VH polynucleotide sequence is encompassed.

5D6.F6.D2 VH polynucleotide sequence:
caggtccagctgcagcagcctggggctgagcttgtgaagcctggggcttc
agtgaagatgtcctgcaaggcttctggctacaccttcaccagcgactgga
taacctgggtgaagcagaggcctggacaaggccttgagtggattggagat
atttatcctcgtagtggtagtactaactacaatgagaagttcaagagcaa
ggccacactgactgtagatatatcctccaacacagcctacatgcagctca
gcagcctgacatctgaggactctgcggtcttttactgtgcaagagggact
tactacggtagtaggtcctactactttgactactggggccaaggcaccac
tctcacagtctcctcag.

By “5D6.F6.D2 VL polynucleotide” is meant a nucleic acid molecule (e.g., DNA) encoding a variable region of the light chain of the 5D6.F6.D2 antibody. In embodiments, a polynucleotide having at least 80%, at least 85%, at least 90%, at least 93%, at least 95%, at least 98%, at least 99% or 100% amino acid sequence identity to the 5D6.F6.D2 VL polynucleotide sequence is encompassed.

5D6.F6.D2 VL polynucleotide sequence:
caaattgttctcacccagtctccagcaatcatgtctgcatctccagggga
gaaggtcaccatgacctgcagtaccagctcaagtgtaagttacatgttct
ggtaccagcagaagccaggatcctcccccagactcctgatttatgacaca
tccaacctggcttctggagtccctgttcgcttcagtggcagtgggtctgg
gacctcttactctctcacaatcagccgaatggaggctgaagatgctgcca
cttattactgccggcagtggagtagttaccccctcacgttcggtgctggg
accaagctggagctgaaac.

The term “antigen-binding fragment,” as used herein, refers to one or more portions or fragments of an antibody that retain the ability to specifically bind to a target antigen. In an embodiment, the target antigen is an HbG-Makassar variant polypeptide or peptide (HbG-Makassar polypeptide or peptide). The antigen-binding function of an antibody can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed by the term “antigen-binding fragment” of an antibody include, but are not limited to: (i) a Fab fragment, a monovalent fragment consisting of the V_{L}, V_{H}, C_{L}, and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). Such antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some cases, by chemical peptide synthesis procedures known in the art. In some embodiments, antigen-binding fragments (e.g., g., Fab′, F(ab′)₂, Fab, scFab, Fv, rlgG, and scFv fragments) of an anti-HbG-Makassar antibody, which are joined by a synthetic linker, are encompassed herein.

By “alteration” is meant a change (increase or decrease) in the structure, expression levels or activity of a gene or polypeptide as detected by standard art known methods such as those described herein. As used herein, an alteration includes a 10% change in expression or activity levels, a 25% change, a 40% change, and a 50% or greater change in expression or activity levels. In embodiments, an alteration is a change in the sequence of a polypeptide or polynucleotide relative to a reference sequence.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide.

Such biochemical modifications could increase the analog's protease resistance, membrane permeability, or half-life, without altering, for example, ligand binding. An analog may include an unnatural amino acid. In addition, analogs of antibodies that retain or enhance the activity of the original antibody, i.e., an anti-HbG-Makassar antibody, are encompassed.

A “binding polypeptide” refers to a polypeptide, or an antigen binding portion or fragment thereof, that has specificity for and specifically binds to the HbG-Makassar polypeptide. In an embodiment, a binding polypeptide is an anti-HbG-Makassar antibody or immunoglobulin or an antigen binding portion or fragment thereof.

As used herein, and without intending to be limiting, a cell preparation, or a tissue or organ preparation, refers to a cell lysate, homogenate, extract (e.g., fluid extract), supernatant, and the like that results after treating the cell, tissue, or organ with an agent or agents that disrupt the cell membrane such that the internal contents of the cell are available for assay. Disruption of the integrity of the cell membrane may occur by chemical, enzymatic, or physical means, e.g., strong detergents, homogenization, or high-energy sound waves. A fluid containing the contents of lysed cells is called a lysate. Cell lysis is used to break open cells to avoid shear forces that would denature or degrade sensitive proteins and DNA. Cell lysis is used (e.g., in western and Southern blotting) to analyze and assess specific proteins, lipids, and/or nucleic acids, as well as in reporter assays, immunoassays, and protein purification methods. Depending upon the detergent used, either all or some membranes in cells are lysed. It will be understood that tissue and organ preparations contain cells that comprise them.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

As used herein, the term “complementarity determining region” (CDR) refers to a hypervariable region found both in the light chain and the heavy chain variable domains ((VL and VH domains, respectively). The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The variable domains of native heavy and light chains each comprise four framework regions (FR1, FR2, FR3, FR4) that primarily adopt a beta-sheet configuration, connected by three CDRs (CDR1, CDR2, CDR3), which form loops that connect, and in some cases form part of, the beta-sheet structure. The CDRs in each chain are held together in close proximity by the FR regions in the order FR1-CDR1-FR2-CDR2-FR3-CDR3-FR4. and the CDRs in each antibody chain contribute to the formation of the target binding site of antibodies (see Kabat et al, Sequences of Proteins of Immunological Interest (National Institute of Health, Bethesda, Md. 1987; incorporated herein by reference). As used herein, numbering of immunoglobulin amino acid residues is done according to the immunoglobulin amino acid residue numbering system of Kabat et al, unless otherwise indicated.

“Detect” refers to identifying the presence, absence or amount of the analyte to be detected. In some embodiments, the analyte is an antigen, epitope, or fragment thereof. In one embodiment, the term “detect” refers to detecting antibody binding to an agent of interest.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. In some embodiments, an antibody as described herein is directly or indirectly linked to a detectable label.

By “disease” is meant any condition or disorder that damages or interferes with the normal function of a cell, tissue, or organ. Examples of diseases include hemoglobinopathies and sickle cell anemia/sickle cell disease (SCD).

By “effective amount” is meant the amount of an agent required to ameliorate the symptoms of a disease relative to an untreated patient. The effective amount of active compound(s) used to practice methods for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In some cases, an effective amount is an amount of a binding polypeptide or an antibody (or an antigen binding portion or fragment thereof) that is needed to perform an activity or function of the binding polypeptide or the antibody or an antigen binding portion or fragment thereof, such as binding to the antigen, e.g., to detect and/or identify the antigen. In an embodiment, the antigen is HbG-Makassar. In embodiments, the binding molecule or antibody is an anti-HbG-Makassar binding molecule or antibody described herein, or an antigen binding portion or fragment thereof.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “framework region” or “FR region” includes amino acid residues that are adjacent to the CDRs. FR region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein, the term “fusion protein” or simply “fusion” refers to a protein that is joined via a covalent bond to another molecule. A fusion protein can be chemically synthesized by, e.g., an amide-bond forming reaction between the N-terminus of one protein to the C-terminus of another protein. Alternatively, a fusion protein containing one protein covalently bound to another protein can be expressed recombinantly in a cell (e.g., a eukaryotic cell or prokaryotic cell) by expression of a polynucleotide encoding the fusion protein, for example, from a vector or the genome of the cell. A fusion protein may contain one protein that is covalently bound to a linker, which in turn is covalently bound to another molecule. Examples of linkers that can be used for the formation of a fusion protein include peptide-containing linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., 2012, Bioorg. Med. Chem., 20:571-582). Fusion proteins can be recombinantly expressed using methods and sequences that are known in the art and described herein.

As used herein, the term “human antibody” refers to an antibody in which substantially every part of the protein (e.g., CDR, framework, C_{L}, C_{H} domains (e.g., C_{H1}, C_{H2}, C_{H3}), hinge, (V_{L}, V_{H})) is substantially non-immunogenic in humans, with only minor sequence changes or variations. A human antibody can be produced in a human cell (e.g., by recombinant expression), or by a non-human animal or a prokaryotic or eukaryotic cell (e.g., yeast) that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598; incorporated by reference herein.

As used herein, the term “humanized” antibodies refers to forms of non-human (e.g., murine) antibodies that are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will comprise substantially all of at least one, and typically two, variable domains, in which all or substantially all of the CDR regions correspond to those of a non-human immunoglobulin. All or substantially all of the FR regions may also be those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos. 5,530,101; 5,585,089; 5,693,761; 5,693,762; and U.S. Pat. No. 6,180,370 to Queen et al; EP239400; PCT publication WO 91/09967; U.S. Pat. No. 5,225,539; EP592106; and EP519596; incorporated herein by reference.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “protein,” “peptide,” “polypeptide,” and their grammatical equivalents are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide can refer to an individual protein or a collection of proteins. One or more of the amino acids in a protein, peptide, or polypeptide can be modified, for example, by the addition of a chemical entity such as a carbohydrate group, a hydroxyl group, a phosphate group, a farnesyl group, an isofarnesyl group, a fatty acid group, a linker for conjugation, functionalization, or other modifications, etc. A protein, peptide, or polypeptide can also be a single molecule or can be a multi-molecular complex. A protein, peptide, or polypeptide can be just a fragment of a naturally occurring protein or peptide. A protein, peptide, or polypeptide can be naturally occurring, recombinant, or synthetic, or any combination thereof.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of some aspects and embodiments is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography.

The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of some aspects and embodiments herein is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of some aspects and embodiments that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of some aspects and embodiments herein. An isolated polypeptide of some aspects and embodiments herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, the term “operatively linked” in the context of a polynucleotide fragment is intended to mean that the two polynucleotide fragments are joined such that the amino acid sequences encoded by the two polynucleotide fragments remain in-frame.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In an embodiment, the reference is a wild-type or healthy cell. In other embodiments and without limitation, a reference is an untreated cell that is not subjected to a test condition, or is subjected to placebo or normal saline, medium, buffer, and/or a control vector that does not harbor a polynucleotide or produce a polypeptide of interest.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, more at least about 25 amino acids, and even more preferably about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, and about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

A “sample” or “biological sample” refers to specimen obtained, taken, generated, or derived from a subject or individual, such as a patient. The specimen may be a body fluid, such as blood, plasma, serum, saliva, sputum, tears, urine; other body fluids, e.g., bronchial fluid, lavage fluid, CNS fluid; stool; cells; tissues; organs (e.g., spleen); and the like. In an embodiment, the sample is an erythroid cell sample, an SCD erythroid cell (i.e., an HbS- or HbG-S-expressing cell) sample. In an embodiment, the cell is a bone marrow cell, a bone marrow derived cell, a stem cell, or a progenitor cell. In an embodiment, the cell is a CD34+ cell (stem cell), e.g., derived from blood (e.g., umbilical cord blood) or bone marrow. In an embodiment, the cell is a human cell. In embodiments, the cells are primary cells or are cultured cells.

As used herein, the term “scFv” refers to a single-chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019, Flo et al., (Gene 77:51, 1989); Bird et al., (Science 242:423, 1988); Pantoliano et al., (Biochemistry 30:10117, 1991); Milenic et al., (Cancer Research 51:6363, 1991); and Takkinen et al., (Protein Engineering 4:837, 1991). The VL and VH domains of a scFv molecule can be derived from one or more antibody molecules. It will also be understood by one of ordinary skill in the art that the variable regions of the scFv molecules of some aspects and embodiments herein can be modified such that they vary in amino acid sequence from the antibody molecule from which they were derived. For example, in one embodiment, nucleotide or amino acid substitutions leading to conservative substitutions or changes at amino acid residues can be made (e.g., in CDR and/or framework residues). Alternatively, or in addition, mutations are made to CDR amino acid residues to optimize antigen binding using art recognized techniques. scFv fragments are described, for example, in WO 2011/084714; incorporated herein by reference.

By “specifically binds” is meant a polypeptide or antibody, or antigen binding fragment or portion thereof, that recognizes and binds to a polypeptide (antigen) of interest (e.g., an HbG-Makassar polypeptide or peptide thereof), but which does not substantially recognize and bind to other molecules in a sample, for example, a biological sample that naturally includes HbG-Makassar polypeptide or peptide and/or other types of hemoglobin polypeptides. In some cases, and without intending to be limiting, an antibody or antigen-binding fragment thereof that specifically binds to an antigen will bind to the antigen with a K_{D} of less than 100 nM. For example, an antibody or antigen-binding fragment thereof that specifically binds to an antigen will bind to the antigen with a K_{D} of up to 100 nM (e.g., between 1 pM and 100 nM). An antibody or antigen-binding fragment thereof that does not exhibit specific binding to a particular antigen or epitope thereof will exhibit a K_{D} of greater than 100 nM (e.g., greater than 500 nm, 1 uM, 100 uM, 500 uM, or 1 mM) for that particular antigen or epitope thereof. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

Nucleic acid molecules (polynucleotides) in some aspects and embodiments herein include any nucleic acid molecule that encodes a polypeptide of some aspects and embodiments herein or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of some aspects and embodiments herein include any nucleic acid molecule that encodes a polypeptide of some aspects and embodiments herein, or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987)Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.10% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In one embodiment, such a sequence is at least 60%, 80% or 85%, 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. In an exemplary approach to determining the degree of identity, a BLAST program may be used, with a probability score between e⁻³ and e⁻¹⁰⁰ indicating a closely related sequence. COBALT is used, for example, with the following parameters:
a) alignment parameters: Gap penalties-11, -1 and End-Gap penalties-5, -1,
b) CDD Parameters: Use RPS BLAST on; Blast E-value 0.003; Find Conserved columns and Recompute on, and
c) Query Clustering Parameters: Use query clusters on; Word Size 4; Max cluster distance 0.8; Alphabet Regular.
   EMBOSS Needle is used, for example, with the following parameters:
a) Matrix: BLOSUM62;
b) GAP OPEN: 10;
c) GAP EXTEND: 0.5;
d) OUTPUT FORMAT: pair;
e) END GAP PENALTY: false;
f) END GAP OPEN: 10; and
g) END GAP EXTEND: 0.5.

By “subject” is meant a mammal, including, but not limited to, a human or non-human mammal, such as a bovine, equine, canine, ovine, or feline mammal. Other mammals include, without limitation, non-human primates (monkeys and the like), mice, rats, rabbits, guinea pigs, gerbils, llamas and alpacas.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “transfecting” or “transfection” is used synonymously and according to some aspects and embodiments herein means the introduction of heterologous nucleic acid (DNA/RNA) into a eukaryotic cell, in particular yeast cells. According to some aspects and embodiments herein, antibody fragments are understood as meaning functional parts of antibodies, such as Fc, Fab, Fab′, Fv, F(ab′)₂, scFv.

According to some aspects and embodiments herein, corresponding biologically active fragments are to be understood as meaning those parts of antibodies which are capable of binding to an antigen, such as Fab, Fab′, Fv, F(ab′)₂, and scFv.

As used herein the term “variable region CDR” includes amino acids in a CDR or complementarity determining region as identified using sequence or structure based methods. As used herein, the term “CDR” or “complementarity determining region” refers to the noncontiguous antigen-binding sites found within the variable regions of both heavy and light chain polypeptides. These particular regions have been described by Kabat et al., J. Biol. Chem. 252:6609-6616, 1977 and Kabat, et al., Sequences of Proteins of Immunological Interest, Fifth Edition, U.S. Department of Health and Human Services, NIH Publication No. 91-3242, 1991; by Chothia et al., (J. Mol. Biol. 196:901-917, 1987), and by MacCallum et al., (J Mol. Biol. 262:732-745, 1996) where the definitions include overlapping or subsets of amino acid residues when compared against each other. In certain embodiments, the term “CDR” is a CDR as defined by Kabat based on sequence comparisons.

As used herein, the term “vector” refers to a means of introducing a nucleic acid sequence into a cell, resulting in a transformed cell. Vectors include plasmids, transposons, phages, viruses, liposomes, and episomes. “Expression vectors” are nucleic acid sequences comprising the nucleotide sequence to be expressed in the recipient cell. Expression vectors contain a polynucleotide sequence as well as additional nucleic acid sequences to promote and/or facilitate the expression of the introduced sequence, such as start, stop, enhancer, promoter, and secretion sequences, into the genome of a mammalian cell. Examples of vectors include nucleic acid vectors, e.g., DNA vectors, such as plasmids, RNA vectors, viruses or other suitable replicons (e.g., viral vectors). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Certain vectors that can be used for the expression of antibodies and antibody fragments of some aspects and embodiments herein include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors of some aspects and embodiments herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “VH” refers to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain of a native antibody has at the amino terminus a variable domain (VH) followed by a number of constant domains. Each light chain of a native antibody has a variable domain at the amino terminus (VL) and a constant domain at the carboxy terminus.

Unless specifically stated or obvious from context, as used herein, the term “or” is understood to be inclusive. Unless specifically stated or obvious from context, as used herein, the terms “a”, “an”, and “the” are understood to be singular or plural.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, such as within 5-fold or within 2-fold, of a value. Where particular values are described in the application and claims, unless otherwise stated the term “about” meaning within an acceptable error range for the particular value should be assumed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A and 1B provide immunoblots. FIG. 1A shows an immunoblot (Western blot) showing the binding specificity of several different anti-HbG-Makassar polypeptide variant antibodies to Makassar globin (HbG) as described herein. The immunoblot was generated by the use of an automated, capillary-based system in which the assay steps, including protein separation, immunoprobing, detection and analysis were fully automated (ProteinSimple Jess protein analysis system, Bio-Techne, MN). The following anti-HbG Makassar antibodies, produced by hybridoma clones as described herein, were assayed (in order from left to right on the blot): 1C10.C1.C7, 1C10.E3.G7, 5D6.G6.G5, and 5D6.F6.D2. Each of the antibodies was analyzed for binding to protein/peptide or samples containing the following proteins or peptides: (1) HbSS protein (commercially available HbS protein (Sigma Aldrich Cat H0392); (2) wildtype β-globin (WT), i.e., lysate from erythroid cells differentiated in vitro from a patient having WT β-globin and base-edited with mRNA encoding a base editor and guide RNA to induce the expression of fetal hemoglobin (gamma globulin); (3) MC109 (“109”) unedited, i.e., lysate from erythroid cells differentiated in vitro from a patient having sickle cell hemoglobin (HbSS, HbS, or HbG-S). The 109 lysate contains only native HbS proteins in a human cell context; (4) MC109 (“109”) edited, i.e., cells from the same HbSS donor that were subjected base editing as described herein, resulting in the production of a functional Makassar beta-globin (HbG) by the edited cells. The production of HbG beta-globin in the cells after the base-editing process was confirmed using mass spectrometry; (5) MKSR globin, i.e., recombinant Makassar globin (HbG) peptide produced in E. coli and used as immunogen in the production of the hybridoma cell lines that generated anti-HbG Makassar antibodies. As observed in FIG. 1A, the anti-HbG Makassar antibodies were found to bind to Makassar globin (HbG) either from the lysates of base-edited HbS cells that expressed Makassar globin after editing or to recombinant HbG Makassar peptide. The anti-HbG Makassar antibodies did not bind to or cross-react with wildtype beta globin or sickle globin (HbS) proteins. Of note, the lack of binding of the anti-HbG Makassar antibodies to the WT globin protein/peptide demonstrates that these antibodies are specific for HbG Makassar protein/peptide and that they detect neither wildtype (WT) beta globin nor fetal hemoglobin (gamma globin) protein/peptide. FIG. 1B shows a second immunoblot in which each of the anti-HbG Makassar monoclonal antibodies 1C10.C1.C7, 1C10.E3.G7, 5D6.G6.G5, and 5D6.F6.D2 (in order from left to right on the blot) was assayed for binding only to the MC109 sample lysates as described for FIG. 1A. The MC109 samples tested were either not base-edited/unedited (“109 UE”) or base-edited (“109 Edit”). As described for FIG. 1A, each of the anti-HbG Makassar antibodies analyzed in FIG. 1B was demonstrated to bind specifically to the HbG Makassar globin protein present in lysates obtained from the base-edited cells (109 Edit). By contrast, no binding of the anti-HbG Makassar antibodies to protein obtained from lysates of unedited cells (109 UE) was detected.

FIGS. 2A and 2B depict a schematic of a binding assay and the assays results. FIG. 2A illustrates the schematic of an immunoassay developed to assess and detect the specificity of binding of anti-HbG Makassar antibodies to HbG Makassar polypeptide, WT globin, or sickle cell globin (HbSS or HbS) in cell lysate samples as described above for FIGS. 1A and 1B. The assay combines electrochemiluminescence and multiarray technology for detection of multiple proteins in a single sample, e.g., a multiplex assay. The assays are typically sandwich-based immunoassays, which use a Multi-Spot microplate, where each spot on a solid substrate (e.g., a well of a microtiter plate) is coated with a unique capture protein, such as a monoclonal antibody.

FIG. 2A provides an illustration of the multi-spot (sandwich-type) assay, and the order of capture and detection antibodies used to detect binding of the antibodies (e.g., anti-HbG Makassar antibody as described herein; anti-WT β-globin (HbB) antibody, or anti-sickle cell globin (HbS) antibody) to protein antigen, i.e., purified protein (e.g., recombinant HbG Makassar peptide, WT β-globin (HbB), (e.g., available from LSBio, Seattle, WA), or sickle cell globin (HbS)), (e.g., available from Rockland Immunochemicals, Inc., Pottstown, PA), or to protein present in cell lysates prepared from base-edited or unedited cells as described supra. The anti-HbG Makassar monoclonal antibodies 1C10.C1.C7 (“C7”), 1C10.E3.G7 (“E367”), 5D6.F6.D2 (“F602”) and 5D6.G6.G5 (“G6”), (“HbG-M mouse mAb”), mouse anti-HbS monoclonal antibody (“HbS mouse mAb”) and mouse anti-HbB monoclonal antibody (“HbB mouse mAb”) were used as capture antibodies in the assay, and the binding specificity of the HbG-M mouse mAbs to purified protein or proteins present in cell lysates of base-edited and unedited cells was assessed relative to the binding of control antibodies, namely, anti-WT β-globin (HbB) antibody and anti-sickle cell globin (HbS) antibody to these same protein antigens. In FIG. 2A, “protein antigen” refers to either purified protein/peptide or proteins present in base-edited or unedited cell lysates used to detect antibody binding in the assay. Second antibodies to the protein antigens were conjugated with a sulfo tag (diamond shape) and were used as detection antibodies in the assay.

FIG. 2B reflects the plate map and the readout of the multiplex assay depicted in FIG. 2A. The top chart shows the binding specificity readout of the C7, E367, F602, and G6 anti-HbG Makassar (“Mksr”/“HbM”) monoclonal antibodies designated above to purified HbG Makassar protein (peptide) antigen, purified HbSS sickle cell protein (peptide) antigen, or purified WT beta-globin protein (peptide) antigen. The bottom chart in FIG. 2B shows the binding specificity readout of the C7, E367, F602, and G6 anti-HbG Makassar monoclonal antibodies designated above to HbG Makassar protein (peptide) antigen, HbSS sickle cell protein (peptide) antigen, or purified WT beta-globin protein (peptide) antigen (HbB) present in cell lysates prepared from base-edited or unedited cells. The higher the numerical value, the stronger the signal, indicating a high level of binding specificity of the anti-HbG Makassar monoclonal antibodies to purified HbG Makassar protein (peptide) antigen and a high level of binding specificity of the anti-HbG Makassar monoclonal antibodies to HbG Makassar protein produced in base-edited cells that normally synthesize sickle cell globin HbSS (HbS). Binding of the anti-HbG Makassar monoclonal antibodies to WT HbB or to sickle cell HbSS was essentially undetectable. As demonstrated by the results of this multiplex assay, all four of the anti-HbG Makassar monoclonal antibodies specifically recognized and bound to HbG Makassar protein.

FIGS. 3A-3C present protein structure models created using the VH and VL amino acid sequences of the anti-HbG Makassar antibodies described herein and a peptide including amino acids 1-19 of the HbG Makassar polypeptide. In particular, three-dimensional (3D) protein structures were predicted and generated based on the VH and VL amino acid sequences of anti-HbG Makassar antibodies 1C10.E3.G7, 1C10.C1.C7, and 5D6.F6.D2 as shown in Table 4 below, and the HbG Makassar peptide, which constitutes amino acids 1-19 of the HbG Makassar polypeptide using the computational neural network-based model AlphaFold. (See, e.g., A. David et al., 2022, J. Mol. Biol., 434(2): 167336; J. Jumper et al., 2021, Nature, 596, 583-589; R. Evans et al., 2022, DeepMind, (doi.org/10.1101/202110.04.463034). FIG. 3A shows a 3D model of the interaction between the VH and VL chains of the anti-HbG Makassar antibody 1C10.E3.G7 and the HbG Makassar peptide. FIG. 3B shows a 3D model of the interaction between the VH and VL chains of the anti-HbG Makassar antibody 1C10.C1.C7 and the HbG Makassar peptide. FIG. 3C shows a 3D model of the interaction between the VH and VL chains of the anti-HbG Makassar antibody 5D6.F6.D2 and the HbG Makassar peptide.

### DETAILED DESCRIPTION OF THE EMBODIMENTS

Featured and described herein are binding polypeptides (proteins), e.g., antibodies, or antigen binding portions or fragments thereof, that specifically bind to the hemoglobin (Hb) variant polypeptide, HbG-Makassar (“Mksr”). In an embodiment, the HbG-Makassar-binding polypeptide is an antibody or an antigen-binding portion or fragment thereof. In an embodiment, the HbG-Makassar-binding polypeptide is a monoclonal antibody or an antigen-binding portion or fragment thereof. The terms “HbG-Makassar,” “HbG-Makassar variant,” HbG-Makassar variant polypeptide/peptide, “HbG-Makassar polypeptide variant,” or “HbG-Makassar polypeptide” are used interchangeably herein. In an embodiment, the binding polypeptides that specifically bind to the HbG-Makassar variant polypeptide or peptide are antibodies, e.g., monoclonal antibodies, or antigen-binding portions or fragments thereof, and are interchangeably termed “anti-Makassar antibodies,” “anti-Hb Makassar antibodies,” or “anti-HbG-Makassar antibodies” herein.

Antibodies that specifically bind to the HbG-Makassar variant polypeptide or peptide were generated. Complementarity determining region 1-3 (CDR1-3) sequences, Framework region 1-4 (FRT-4) sequences, Heavy chain variable region (VH) and Light chain variable region (VL) amino acid sequences, and polynucleotide (e.g., DNA) sequences encoding the VH and VL sequences of representative anti-HbG Makassar monoclonal antibodies (generated from hybridoma clones) are provided in the following Tables 1-5:

TABLE 1

| Anti-HbG | | | | | | |
|---|---|---|---|---|---|---|
| Makassar | | | | | | |
| Antibody Clone | | | | | VL | |
| Designation | VH CDR1 | VH CDR2 | VH CDR3 | VL CDR1 | CDR2 | VL CDR3 |
| 1C10.E3.G7 | GIDFSRYW | INIDSSTI | ARAYDGYSLDY | SSVSY | DTS | RQWSSYPLT |
| 1C10.C1.C7 | GYTFTNYF | INPKNGGI | ARGSANWGAY | QRTNC | HDL | QQWSSYPLT |
| 5D6.F6.D2 | GYTFTSDW | IYPRSGST | ARGTYYGSRSYYFDY | SSVSY | DTS | RQWSSYPLT |

TABLE 2

| Anti-HbG | | | | |
|---|---|---|---|---|
| Makassar | | | | |
| Antibody | | | | |
| Clone | | | | |
| Designa- | | | | |
| tion | VH FR1 | VH FR2 | VH FR3 | VH FR4 |
| 1C10.E3.G7 | EVQLQESGGGLVQPG | MSWVRRAPGKGLEWIGE | NYAPSLKDKFIISRDNAKN | WGQGTSVTVSS |
| | GSLKLSCAAS | | TLYLQMSKVRSEDTALYYC | |
| 1C10.C1.C7 | EVLLQQSGPELVKPG | MNWVKQSHGKSLEWIGD | SYNQKFKGKATLIVDKSSS | WGQGTLVTVSA |
| | ASVKISCKAS | | TAYMELRSLTSEDSAVYYC | |
| 5D6.F6.D2 | QVQLQQPGAELVKPG | ITWVKQRPGQGLEWIGD | NYNEKFKSKATLTVDISSN | WGQGTTLTVSS |
| | ASVKMSCKAS | | TAYMQLSSLTSEDSAVFYC | |

TABLE 3

| Anti-HbG | | | | |
|---|---|---|---|---|
| Makassar | | | | |
| Antibody | | | | |
| Clone | | | | |
| Designa- | | | | |
| tion | VL FR1 | VL FR2 | VL FR3 | VL FR4 |
| 1C10. | QIVLTQS | MFWYQQK | NLASGVP | FGAGT |
| E3.G7 | PAIMSAS | PGSSPRL | VRFSGSG | KLELK |
| | PGEKVTM | LIY | SGTSYSL | |
| | TCSTS | | TISRMEA | |
| | | | EDAATYY | |
| | | | C | |
| 1C10. | WWEDGYS | SRPVSSN | QCQLKFP | FGAGT |
| C1.C7 | WCSISHF | HVCISRG | VRFSGSG | KLELK |
| | QLPANQC | EGH | SGTSYSL | |
| | LSHTV | | TISRMEA | |
| | | | EDAATYY | |
| | | | C | |
| 5D6. | QIVLTQS | MFWYQQK | NLASGVP | FGAGT |
| F6.D2 | PAIMSAS | PGSSPRL | VRFSGSG | KLELK |
| | PGEKVTM | LIY | SGTSYSL | |
| | TCSTS | | TISRMEA | |
| | | | EDAATYY | |
| | | | C | |

TABLE 4

| Anti-HbG | | |
|---|---|---|
| Makassar | VH | VL |
| Antibody | Amino | Amino |
| Clone | Acid | Acid |
| Designation | Sequence | Sequence |
| 1C10.E3.G7 | EVQLQESGGG | QIVLTQSPAI |
| | LVQPGGSLKL | MSASPGEKVT |
| | SCAASGIDFS | MTCSTSSSVS |
| | RYWMSWVRRA | YMFWYQQKPG |
| | PGKGLEWIGE | SSPRLLIYDT |
| | INIDSSTINY | SNLASGVPVR |
| | APSLKDKFII | FSGSGSGTSY |
| | SRDNAKNTLY | SLTISRMEAE |
| | LQMSKVRSED | DAATYYCRQW |
| | TALYYCARAY | SSYPLTFGAG |
| | DGYSLDYWGQ | TKLELK |
| | GTSVTVSS | |
| 1C10.C1.C7 | EVLLQQSGPE | WWEDGYSWCS |
| | LVKPGASVKI | ISHFQLPANQ |
| | SCKASGYTFT | CLSHTVQRTN |
| | NYFMNWVKQS | CSRPVSSNHV |
| | HGKSLEWIGD | CISRGEGHHD |
| | INPKNGGISY | LQCQLKFPVR |
| | NQKFKGKATL | FSGSGSGTSY |
| | IVDKSSSTAY | SLTISRMEAE |
| | MELRSLTSED | DAATYYCQQW |
| | SAVYYCARGS | SSYPLTFGAG |
| | ANWGAYWGQG | TKLELK |
| | TLVTVSA | |
| 5D6.F6.D2 | QVQLQQPGAE | QIVLTQSPAI |
| | LVKPGASVKM | MSASPGEKVT |
| | SCKASGYTFT | MTCSTSSSVS |
| | SDWITWVKQR | YMFWYQQKPG |
| | PGQGLEWIGD | SSPRLLIYDT |
| | IYPRSGSTNY | SNLASGVPVR |
| | NEKFKSKATL | FSGSGSGTSY |
| | TVDISSNTAY | SLTISRMEAE |
| | MQLSSLTSED | DAATYYCRQW |
| | SAVFYCARGT | SSYPLTFGAG |
| | YYGSRSYYFD | TKLELK |
| | YWGQGTTLTV | |
| | SS | |

TABLE 5

| Anti-HbG | | |
|---|---|---|
| Makassar | VH | VL |
| Antibody | Polynucleotide | Polynucleotide |
| Clone | Sequence | Sequence |
| Designation | (5′ to 3′) | (5′ to 3′) |
| 1C10.E3.G7 | gaggtgcagc | caaattgttc |
| | tgcaggagtc | tcacccagtc |
| | tggaggtggc | tccagcaatc |
| | ctggtgcagc | atgtctgcat |
| | ctggaggatc | ctccagggga |
| | cctgaaactc | gaaggtcacc |
| | tcctgtgcag | atgacctgca |
| | cctcaggaat | gtaccagctc |
| | cgattttagt | aagtgtaagt |
| | agatactgga | tacatgttct |
| | tgagttgggt | ggtaccagca |
| | tcgggggctc | gaagccagga |
| | cagggaaagg | tcctccccca |
| | actagaatgg | gactcctgat |
| | attggagaaa | ttatgacaca |
| | ttaatataga | tccaacctgg |
| | tagcagtaca | cttctggagt |
| | ataaactatg | ccctgttcgc |
| | caccatctct | ttcagtggca |
| | aaaggataaa | gtgggtctgg |
| | ttcatcatct | gacctcttac |
| | ccagagacaa | tctctcacaa |
| | cgccaaaaat | tcagccgaat |
| | acgctgtacc | ggaggctgaa |
| | tgcaaatgag | gatgctgcca |
| | caaagtgaga | cttattactg |
| | tctgaggaca | ccggcagtgg |
| | cagcccttta | agtagttacc |
| | ttactgtgca | ccctcacgtt |
| | agggcctatg | cggtgctggg |
| | atggttattc | accaagctgg |
| | gttggactac | agctgaaac |
| | tggggtcaag | |
| | gaacctcagt | |
| | caccgtctcc | |
| | tcag | |
| 1C10.C1.C7 | gaggtcctgc | tggtgggaag |
| | tgcaacaatc | atggatacag |
| | tggacctgag | ttggtgcagc |
| | ctggtgaagc | atcagccatt |
| | ctggggcttc | ttcagcttcc |
| | agtgaagatt | tgctaatcag |
| | tcctgtaagg | tgcctcagtc |
| | cttctggata | atactgtcca |
| | cacgttcact | gaggacaaat |
| | aactacttca | tgttctcgcc |
| | tgaactgggt | cagtctccag |
| | gaagcagagc | caatcatgtc |
| | catggaaaga | tgcatctcca |
| | gccttgagtg | ggggagaagg |
| | gattggagat | tcaccatgac |
| | attaatccta | ctgcagtgcc |
| | agaatggtgg | agctcaagtt |
| | tattagttac | ccctgttcgc |
| | aaccagaaat | ttcagtggca |
| | ttaagggcaa | gtgggtctgg |
| | ggccacattg | gacctcttac |
| | attgtagaca | tctctcacaa |
| | agtcctccag | tcagccgaat |
| | cacagcctac | ggaggctgaa |
| | atggagctcc | gatgctgcca |
| | gcagcctgac | cttattactg |
| | ttctgaggac | ccagcagtgg |
| | tctgcagtct | agtagttacc |
| | attattgtgc | ccctcacgtt |
| | aagagggtca | cggtgctggg |
| | gctaactggg | accaagctgg |
| | gggcttactg | aactgaaac |
| | gggccaaggg | |
| | actctggtca | |
| | ctgtctctgc | |
| | ag | |
| 5D6.F6.D2 | caggtccagc | caaattgttc |
| | tgcagcagcc | tcacccagtc |
| | tggggctgag | tccagcaatc |
| | cttgtgaagc | atgtctgcat |
| | ctggggcttc | ctccagggga |
| | agtgaagatg | gaaggtcacc |
| | tcctgcaagg | atgacctgca |
| | cttctggcta | gtaccagctc |
| | caccttcacc | aagtgtaagt |
| | agcgactgga | tacatgttct |
| | taacctgggt | ggtaccagca |
| | gaagcagagg | gaagccagga |
| | cctggacaag | tcctccccca |
| | gccttgagtg | gactcctgat |
| | gattggagat | ttatgacaca |
| | atttatcctc | tccaacctgg |
| | gtagtggtag | cttctggagt |
| | tactaactac | ccctgttcgc |
| | aatgagaagt | ttcagtggca |
| | tcaagagcaa | gtgggtctgg |
| | ggccacactg | gacctcttac |
| | actgtagata | tctctcacaa |
| | tatcctccaa | tcagccgaat |
| | cacagcctac | ggaggctgaa |
| | atgcagctca | gatgctgcca |
| | gcagcctgac | cttattactg |
| | atctgaggac | ccggcagtgg |
| | tctgcggtct | agtagttacc |
| | tttactgtgc | ccctcacgtt |
| | aagagggact | cggtgctggg |
| | tactacggta | accaagctgg |
| | gtaggtccta | agctgaaac |
| | ctactttgac | |
| | tactggggcc | |
| | aaggcaccac | |
| | tctcacagtc | |
| | tcctcag | |

### Hemoglobins, Hemoglobin Variants, and Hemoglobinopathies

The alpha (HBA) and beta (HBB) loci determine the structure of the two types of polypeptide chains in adult hemoglobin, Hemoglobin A (Hb A). The normal adult hemoglobin tetramer consists of two alpha (α) chains and two beta (β) chains. Mutant β-globin causes sickle cell anemia. The absence of a β-chain causes β-zero (β0)-thalassemia. Reduced amounts of detectable β-globin causes β-plus (β+)-thalassemia. The order of the genes in the beta-globin gene cluster is 5′-epsilon-gamma-G-gamma-A-delta-beta-3′.

Hemoglobinopathies are inherited abnormalities of globin chain synthesis. Sickle cell disease (SCD), also known as sickle cell anemia, is the most common monogenic blood disease and is associated with the production of the hemoglobin variant, HbS. More than 1000 natural mutations have been reported in human hemoglobin variants. These hemoglobin variants were found to be the result of single amino acid substitutions throughout the gene. The clinical effects of the hemoglobin variants are diverse and range from clinically insignificant to severe forms of hemoglobin disorders. While SCD is most prevalent in sub-Saharan Africa and in parts of the Mediterranean region, the Middle East and the Indian subcontinent, beta-thalassemia is most common among individuals in the Mediterranean, Africa and South Asia. In Southeast Asia, beta-thalassemia affects 0-11% of population.

The HbG-Makassar mutation was first identified in Makassar, Sulawesi (Celebes), Republic of Indonesia. The HbG Makassar variant was also identified in individuals living in Thailand and in Malaysia. (A. S. Mohamad et al., 2018, Hematology Reports, 10:7210, pages 92-95; R. Q. Blackwell, 1970, Biochim Biophys Acta, 396-401; V. Viprakasit. 2002, Hemoglobin, 26:245-53). The electrophoretic mobility of the HbG-Makassar 1-hemoglobin variant was observed to be slower than that of normal (wild-type) β-hemoglobin (β-globin). The HbG-Makassar structural anomaly is at the β-6 or A3 position w % here the glutamyl residue that is normally present in β-globin is replaced by an alanyl residue. The substitution of a single amino acid in the gene encoding β-globin subunit β-6 glutamyl to valine results in sickle cell disease.

### Uses of the HbG-Makassar Variant-Binding Polypeptides and Anti-HbG-Makassar Antibodies

The HbG-Makassar variant binding polypeptides or peptides, and anti-HbG-Makassar antibodies described herein, are advantageous for specifically binding to, detecting, selecting, identifying, and/or isolating an HbG-Makassar variant polypeptide or peptide versus a sickle cell hemoglobin HbS variant polypeptide, (or other hemoglobin protein or peptide), for example, in a biological sample, e.g., a cell. In an embodiment, the sample is obtained from an individual (e.g., a patient undergoing testing or analysis for SCD or other hemoglobinopathy). In an embodiment, the individual is a patient whose cells have been subjected to genomic or base editing, thereby resulting in the production of HbG-Makassar variant polypeptide in the patient's cells.

Under the conditions of gel electrophoresis, e.g., alkaline gel electrophoresis, many variants of the β- and α-globin chains migrate in a manner similar to the HbS variant. In some cases, hemoglobin variants such HbD or HbG can be separated by acid gel electrophoresis; however, this is not the case with HbG-Makassar. In general, the HbG-Makassar variant polypeptide cannot be distinguished from the SCD HbS polypeptide by techniques such as isoelectric focusing, hemoglobin electrophoresis separation by cation-exchange High Performance Liquid Chromatography (HPLC), globin chain electrophoresis, hemoglobin electrophoresis, or cellulose acetate electrophoresis. Because these techniques have been unable to separate the HbG-Makassar and HbS polypeptides, the anti-HbG-Makassar antibodies described herein provide highly beneficial products, reagents and biological tools, which are highly useful for identifying the HbG-Makassar polypeptide and for separating the HbG-Makassar from other hemoglobin polypeptides, such as the HbS (SCD) polypeptide, e.g., in a mixture or sample containing the two, as well as other proteins, by specifically binding to the HbG-Makassar variant polypeptide. In the medical and clinical fields, as well as in the research and biotechnology fields, the use of the HbG-Makassar variant-binding polypeptides and the anti-HbG-Makassar antibodies described herein can prevent and/or alleviate the incorrect identification or the misidentification of the HbG-Makassar and the HbS polypeptides, thereby preventing these two proteins from being incorrectly identified as being the same polypeptides, and preventing a potential misdiagnosis of a patient as having Sickle Cell Disease (SCD).

Accordingly, the HbG-Makassar variant binding polypeptides and anti-HbG-Makassar antibodies described herein, which have virtually no cross-reactivity with other forms of hemoglobin, can be used to detect and identify with specificity and accuracy the HbG-Makassar variant polypeptide or peptide, e.g., in a sample. In embodiments, such reagents and products as described herein are especially useful to determine specifically the presence and/or production of the HbG-Makassar variant polypeptide or peptide in patients treated for SCD via genetic engineering and base editing techniques and therapies. In an embodiment, the use of the HbG-Makassar variant binding polypeptides and anti-HbG-Makassar antibodies described herein for the specific identification of HbG-Makassar is efficient and effective and can complement the use of DNA, LC-MS, or HPLC (e.g., ultra-high-performance liquid chromatography (UPLC)) assays. In an embodiment, the use of the HbG-Makassar variant binding polypeptides and anti-HbG-Makassar antibodies described herein for the specific identification of HbG-Makassar can obviate the need for DNA, LC-MS, HPLC, or UPLC analyses. By way of nonlimiting example, the HbG-Makassar variant binding polypeptides and anti-HbG-Makassar antibodies described herein can be used to determine, detect, screen for, select, and/or identify the HbG-Makassar variant versus the HbS/HbSS (SCD) variant in cells, e.g., cells edited with base editors (such as ABE base editors), see, e.g., U.S. Pat. No. 11,242,760; WO 2021/163587; WO 2021/041945; WO/2019/217942; WO/2020/168051; WO/2020/168075, incorporated fully herein by reference, and below. In addition, the HbG-Makassar variant binding polypeptides and anti-HbG-Makassar antibodies can be used to screen and assess patients to provide specific identification of authentic SCD (HbS) patients versus patients expressing an HbG-Makassar polypeptide. Such uses can alleviate or prevent the misdiagnosis of SCD (HbS) in patients who do not express SCD HbS, but instead express an HbG-Makassar variant or another hemoglobin or globin variant.

In an aspect, the anti-HbG-Makassar binding polypeptides and/or antibodies as described herein, e.g., 1C10.E3.G7, 1C10.C1.C7, or 5D6.F6.D2, or antigen binding portions thereof, can be used in a method of identifying and/or selecting a subject whose cells express HbG-Makassar polypeptide, in which the method involves (a) contacting a preparation (e.g., a lysate, suspension, or supernatant) of a cell sample obtained from the subject with an HbG-Makassar binding polypeptide or and anti-HbG-Makassar antibody, or an antigen binding portion or region thereof as described herein; (b) detecting specific binding between the antibody and the HbG-Makassar polypeptide in the preparation; and identifying and/or selecting the subject as having cells that express HbG-Makassar based on the detecting step (b). In an embodiment, the sample is a cell obtained from the subject. In an embodiment, the cells are obtained or prepared from a tissue or organ of the subject. In an embodiment, the subject is a patient who is undergoing testing for SCD. In embodiments, the cell, tissue, or organ sample is treated or processed (e.g., homogenized) to obtain the lysate, suspension or supernatant (liquid) preparation containing cells.

In an aspect, the anti-HbG-Makassar binding polypeptides and/or antibodies as described herein, e.g., 1C10.E3.G7, 1C10.C1.C7, or 5D6.F6.D2, or antigen binding portions thereof, can be used in a method of monitoring a subject for the production (e.g., prolonged or sustained production) of HbG-Makassar polypeptide, in which the method involves (a) contacting a preparation (e.g., a lysate, suspension, or supernatant) of a cell sample obtained from the subject with an HbG-Makassar binding polypeptide or and anti-HbG-Makassar antibody, or an antigen binding portion or region thereof as described herein at a first time point and detecting specific binding between the polypeptide or the antibody and an HbG-Makassar polypeptide in the preparation; (b) contacting a preparation (e.g., a lysate, suspension, or supernatant) of a cell sample obtained from the subject with an HbG-Makassar binding polypeptide or and anti-HbG-Makassar antibody, or an antigen binding portion or region thereof as described herein at one or more additional time points following step (a) and detecting specific binding between the binding polypeptide or the antibody and an HbG-Makassar polypeptide in the preparation; and (c) monitoring that the subject is expressing the HbG-Makassar polypeptide by detecting the same level or a greater level of the HbG-Makassar polypeptide in the subject's cells in step (b) versus step (a). In embodiments of the above methods, the cells are bone marrow-derived cells, cord blood cells, or red blood cells (erythrocytes). In an embodiment, the sample is a cell obtained from the subject. In an embodiment, the cells are obtained or prepared from a tissue or organ of the subject. In an embodiment, the subject's cells have been genetically edited to express (and produce) the HbG-Makassar polypeptide prior to step (b). In an embodiment, the subject is a patient afflicted with sickle cell disease (SCD) or a hemoglobinopathy that is treatable by the expression (and production) of the HbG-Makassar polypeptide.

It will be understood that if no specific binding is detected in the detecting step(s) of the above methods, then the cells do not express or produce a HbG-Makassar polypeptide.

In an embodiment of the methods, the specific binding (e.g., binding affinity, degree or level of binding) of the HbG-Makassar binding polypeptides and anti-HbG-Makassar antibodies to an HbG-Makassar variant polypeptide or peptide is compared to the binding of a suitable control (or reference) antibody, e.g., an irrelevant or non-cross-reactive control antibody or immunoglobulin, or an antibody that does not recognize or bind to the HbG-Makassar variant. In an embodiment, an antibody that specifically binds to normal hemoglobin, e.g., HbA, β-globin (HbB), or to another hemoglobin variant polypeptide, but does not recognize or specifically bind to the HbG-Makassar variant, may be used as a control or reference.

Any suitable method may be used for detecting HbG-Makassar in the sample using the HbG-Makassar binding polypeptides and anti-HbG-Makassar antibodies described herein, e.g., immunoassay, chip (antigen chip) assay, lateral flow assay, mass spectrometry, etc. Non-limiting examples of immunoassays that may be used include enzyme-linked immunosorbent assay (ELISA), flow cytometry with multiplex beads, surface plasmon resonance (SPR), ellipsometry (an optical technique for investigating the dielectric properties (complex refractive index or dielectric function) of thin films to measure the change of polarization upon reflection or transmission and compared to a model), and other immunoassays that employ, for example, laser scanning, colorimetric or light detecting, photon detecting via a photo-multiplier, photographing with a digital camera based system or video system, radiation counting, fluorescence detecting, luminescence, chemiluminescence, or electrochemiluminescence detecting, electronic detecting, magnetic detecting and any other system that allows quantitative measurement of antigen-antibody binding.

The anti-HbG-Makassar binding molecules and antibodies used in detection methods may be subjected to any desired degree of dilution or purification prior to being tested for their capacity to specifically bind to antigen, namely, HbG-Makassar. The methods can be practiced using whole antibodies, or antigen binding portions or fragments which comprise one or more antibody variable region (VH and/or VL) that recognizes and binds to HbG-Makassar polypeptide.

Diagnostic methods are encompassed herein. As would be appreciated by the skilled practitioner in the art, diagnosis refers to the process of identifying a medical condition or disease (e.g., SCD or other hemoglobinopathies) by certain signs and symptoms, as well as from the results of diagnostic procedures, such as detecting in a biological sample obtained from a patient the presence or absence of an antigen associated or not associated with the condition or disease. In embodiments, the HbG-Makassar binding polypeptides and anti-HbG-Makassar antibodies or antigen binding portions or fragments thereof as described herein are used in such methods to detect the presence (or absence) of the HbG-Makassar variant in a sample obtained from a subject (patient). In embodiments, the HbG-Makassar binding polypeptides and anti-HbG-Makassar antibodies or antigen binding portions or fragments thereof as described herein are used in such methods to determine whether the patient expresses the HbG-Makassar variant polypeptide or peptide, or another hemoglobin polypeptide or peptide, such as HbS (sickle cell hemoglobin) or normal hemoglobin polypeptides or peptides. Diagnosing also encompasses screening for a disease, e.g., SCD or other hemoglobinopathy; detecting a presence or a severity of a disease, distinguishing a disease from other diseases including those diseases that may feature one or more similar or identical symptoms, providing prognosis of a disease, monitoring disease progression or relapse, as well as assessment of treatment efficacy and/or relapse of a disease, disorder or condition, selecting a therapy and/or a treatment for a disease, optimization of a given therapy (dose/schedule) for a disease, monitoring a therapeutic treatment, and/or predicting the suitability of a therapy for specific patients or subpopulations or determining the appropriate dosing of a therapeutic product in patients or subpopulations.

### Use of Anti-HbG-Makassar Antibodies to Detect and Identify an HbG-Makassar β-Globin Variant Following Genetic-Based Treatments for SCD

Genetic techniques have been designed to switch red blood cells (erythrocytes) from making mutant β-globin to producing HbG-Makassar (“the HbG-Makassar variant”), which is a rare, naturally occurring form of β-globin. The presence of the HbG-Makassar variant is associated with a normal phenotype even in individuals carrying two copies.

Direct genomic editing of the mutation, Glu6Val, that causes sickle cell disease has not been possible at high efficiency without causing double strand DNA breaks. Adenine base editors (ABEs) have been shown to precisely make A-T to G-C base pair conversions with low rates of indels and without double strand DNA breaks. (See, e.g., N. M. Gaudelli et al., Nature, 551:464-471 (2017) and N. M. Gaudelli et al., Nature Biotechnol., 38(7):892-900 (2020), both incorporated by reference herein). This results in the conversion of valine to alanine and the production of the naturally occurring HbG-Makassar variant, which presents with normal hematological parameters and red blood cell morphology. Furthermore, alanine substitutions at this residue of the β-hemoglobin subunit did not contribute to polymer formation in vitro. (see, e.g., U.S. Pat. No. 11,242,760).

In particular, ABE variants have been reported that efficiently recognize and edit the sickle mutation, converting the sickle-causing valine to an alanine. (see, e.g., U.S. Pat. No. 11,242,760). This conversion generates a naturally-occurring form of β-globin, namely, the HbG-Makassar polypeptide. This variant was previously identified in asymptomatic homozygous individuals that have normal hematologic parameters and no evidence of hemoglobin polymerization or sickling of red blood cells. It has been reported that the ABE variants could successfully edit human CD34+ cells harboring the sickle trait and be maintained throughout hematopoiesis, especially erythropoiesis. In these studies, the high, bi-allelic editing and conversion to the Makassar β-globin variant successfully reduced HbS globin to a level of <15% and reduced in vitro sickling under hypoxia. The direct editing of the causative sickle cell mutation (HbS) to the naturally occurring and asymptomatic HbG-Makassar provides a significant new treatment paradigm for patients with SCD.

Generation and Screening of Antibodies that Bind to the HbG-Makassar Variant Polypeptide or Peptide

Antibodies, including recombinantly produced antibodies, that specifically bind to the HbG-Makassar variant polypeptide or peptide thereof are provided and described herein. In embodiments, the antibodies are 1C10.E3.G7, 1C10.C1.C7, 5D6.F6.D2, or antigen binding portions thereof, as described herein.

Methods for generating antibodies against a protein or peptide of interest are known and practiced in the art. When animals are immunized with antigens they respond by generating a polyclonal antibody response comprised of many individual monoclonal antibody specificities. It is the sum of these individual specificities that make polyclonal antibodies useful in so many different assays. Individual monoclonal antibodies were originally isolated by immortalizing individual B cells using hybridoma technology (Kohler and Milstein, Nature 256, 495, 2011), in which B cells from an immunized animal are fused with a myeloma cell. With the advent of molecular biology, in vitro methods to generate antibodies against proteins of interest, such as HbG-Makassar, have been developed.

The terms “antigen of interest” or “target protein” are used herein interchangeably and refer generally to the agent recognized and specifically bound by an antibody. In an embodiment, such an antigen of interest or target protein is the HbG-Makassar polypeptide, or an antigenic and/or immunogenic portion thereof.

An antibody is a polypeptide chain-containing molecular structure with a specific shape that specifically binds an epitope, where one or more non-covalent binding interactions stabilize the complex between the molecular structure and the epitope. In one embodiment, an antibody molecule is an immunoglobulin (e.g., IgG, IgM, IgA, IgE, IgD). Antibodies from a variety of sources, e.g. human, rodent, rabbit, cow, sheep, pig, dog, or fowl are considered “antibodies.” Numerous antibody coding sequences have been described; and others may be raised by methods well-known in the art.

For example, antibodies or antigen binding fragments may be produced by genetic engineering. Antibody coding sequences of interest include those encoded by native sequences, as well as nucleic acids that, by virtue of the degeneracy of the genetic code, are not identical in sequence to a wild-type nucleic acid sequence. Variant polypeptides can include amino acid (aa) substitutions, additions or deletions. The amino acid substitutions can be conservative amino acid substitutions or substitutions to eliminate non-essential amino acids, such as to alter a glycosylation site, or to minimize misfolding by substitution or deletion of one or more cysteine residues that are not necessary for function. Variants can be designed so as to retain or have enhanced biological activity of a particular region of the protein (e.g., a functional domain, catalytic amino acid residues). Variants also include fragments of the polypeptides disclosed herein, particularly biologically active fragments and/or fragments corresponding to functional domains. Techniques for in vitro mutagenesis of cloned genes are known. Also included in some aspects and embodiments herein are polypeptides that have been modified using ordinary molecular biological techniques so as to improve their resistance to proteolytic degradation or to optimize solubility properties or to render them more suitable as a therapeutic agent.

Chimeric antibodies may be made by recombinant means by combining the variable light and heavy chain regions obtained from antibody producing cells of one species with the constant light and heavy chain regions from another. Typically chimeric antibodies utilize rodent or rabbit variable regions and human constant regions, in order to produce an antibody with predominantly human domains. The production of such chimeric antibodies is well known in the art, and may be achieved by standard means (as described, e.g., in U.S. Pat. No. 5,624,659, incorporated fully herein by reference).

Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although apparently complex, the process is straightforward in practice. See, e.g., U.S. Pat. No. 6,187,287, incorporated fully herein by reference.

In addition to entire immunoglobulins (or their recombinant counterparts), immunoglobulin fragments comprising the epitope binding site (e.g., Fab′, F(ab′)₂, or other fragments) may be synthesized. “Fragment,” or minimal immunoglobulins may be designed utilizing recombinant immunoglobulin techniques. For instance “Fv” immunoglobulins for use in some aspects and embodiments herein may be produced by synthesizing a variable light chain region and a variable heavy chain region. Combinations of antibodies are also of interest, e.g. diabodies, which comprise two distinct Fv specificities.

Immunoglobulins may be modified post-translationally, e.g., to add chemical linkers, detectable moieties, such as fluorescent dyes, enzymes, substrates, chemiluminescent moieties and the like, or specific binding moieties, such as streptavidin, avidin, or biotin, and the like may be utilized in the methods and compositions of some aspects and embodiments herein.

### Mapping Epitopes of the HbG-Makassar Variant Polypeptide

Anti-HbG-Makassar antibodies, and antigen-binding fragments thereof, can be produced by screening libraries of polypeptides (e.g., antibodies and antigen-binding fragments thereof) for functional molecules that are capable of binding to the HbG-Makassar variant polypeptide or peptide (and/or epitopes within the HbG-Makassar variant polypeptide or peptide) that selectively bind to the HbG-Makassar variant polypeptide or peptide compared with other Hb polypeptides or peptides, e.g., HbS (associated with SCD). Epitopes can be modeled by screening antibodies or antigen-binding fragments thereof against a series of linear or cyclic peptides containing residues that correspond to a desired epitope within the HbG-Makassar variant polypeptide or peptide.

As an example, peptides containing individual fragments isolated from the HbG-Makassar variant polypeptide or peptide that specifically bind to HbG-Makassar and differentiate between the HbG-Makassar polypeptide and an SCD HbS polypeptide (or other hemoglobin protein or peptide) based on such binding specificity can be synthesized by peptide synthesis techniques described herein or known in the art. These peptides can be immobilized on a solid surface and screened for molecules that bind to anti-HbG-Makassar antibodies and antigen-binding fragments thereof, such as representative antibodies 1C10.E3.G7, 1C10.C1.C7, 5D6.F6.D2, 5D6.G6.G5, or antigen binding portions thereof, as described herein, e.g., using an ELISA-based screening platform using established procedures. Using this assay, peptides that specifically bind to the anti-HbG-Makassar antibodies with high affinity therefore contain residues within epitopes of the HbG-Makassar polypeptide antigen that preferentially bind these antibodies. Peptides identified in this manner can be used to screen libraries of antibodies and antigen-binding fragments thereof in order to identify anti-HbG-Makassar antibodies useful in generating the HbG-Makassar antibodies of some aspects and embodiments herein.

Screening of libraries for HbG-Makassar variant-binding polypeptides or peptides Methods for high throughput screening of polypeptide (e.g., antibody or antigen-binding antibody fragment) libraries for molecules capable of binding to the HbG-Makassar variant polypeptide or peptide (and/or epitopes within the HbG-Makassar variant polypeptide or peptide) include, without limitation, display techniques including phage display, bacterial display, yeast display, mammalian display, ribosome display, mRNA display, and cDNA display. The use of phage display to isolate ligands that bind biologically relevant molecules has been reviewed, e.g., in Felici et al. (Biotechnol. Annual Rev. 1:149-183, 1995), Katz (Annual Rev. Biophys. Biomol. Struct. 26:27-45, 1997), and Hoogenboom et al. (Immunotechnology 4:1-20, 1998). Several randomized combinatorial peptide libraries have been constructed to select for polypeptides that bind different targets, e.g., cell surface receptors or DNA (reviewed by Kay (Perspect. Drug Discovery Des. 2, 251-268, 1995), Kay et al., (Mol. Divers. 1:139-140, 1996)). Proteins and multimeric proteins have been successfully phage-displayed as functional molecules (see. e.g., EP 0349578A, EP 4527839A, EP 0589877A; Chiswell and McCafferty (Trends Biotechnol. 10, 80-84 1992)). In addition, functional antibody fragments (e.g. Fab, single-chain Fv [scFv]) have been expressed as reported by McCafferty et al. (Nature 348: 552-554, 1990), Barbas et al. (Proc. Natl. Acad Sci. USA 88:7978-7982, 1991), and Clackson et al. (Nature 352:624-628, 1991). These references are hereby incorporated by reference in their entirety.

In addition to generating HbG-Makassar variant-binding polypeptides (e.g., anti-HbG-Makassar variant-binding polypeptides, antibodies, and antigen-binding fragments thereof) of some aspects and embodiments herein, in vitro display techniques, which are known and practiced in the art, also provide methods for improving the affinity of an anti-HbG-Makassar variant-binding polypeptide, antibody, or antigen-binding fragments thereof. For instance, rather than screening libraries of antibodies and fragments thereof containing completely randomized hypervariable regions, narrower libraries of antibodies and antigen-binding fragments thereof that feature targeted mutations at specific sites within hypervariable regions can be screened. This can be accomplished, for example, by assembling libraries of polynucleotides encoding antibodies or antigen-binding fragments thereof that encode random mutations only at particular sites within hypervariable regions. These polynucleotides can then be expressed in, e.g., filamentous phage, bacterial cells, yeast cells, mammalian cells, or in vitro using, e.g., ribosome display, mRNA display, or cDNA display techniques in order to screen for antibodies or antigen-binding fragments thereof that specifically bind to the HbG-Makassar polypeptide or peptide (and epitopes thereof) with improved binding affinity. Yeast display, for instance, is well-suited for affinity maturation, and has been used previously to improve the affinity of a single-chain antibody to a K_{D} of 48 fM (Boder et al. (Proc Natl Acad Sci USA 97:10701, 2000)).

Additional in vitro techniques that can be used for the generation and affinity maturation of anti-HbG-Makassar variant-binding polypeptides, antibodies, and antigen-binding fragments thereof (e.g., single-chain polypeptides, antibodies, and antigen-binding fragments thereof) of some aspects and embodiments herein include the screening of combinatorial libraries of antibodies or antigen-binding fragments thereof for functional molecules capable of specifically binding to peptides derived from the HbG-Makassar polypeptide. Combinatorial antibody libraries can be obtained, e.g., by expression of polynucleotides encoding randomized hypervariable regions of an antibody or antigen-binding fragment thereof in a eukaryotic or prokaryotic cell. This can be achieved, e.g., using gene expression techniques described herein or known in the art. Heterogeneous mixtures of antibodies can be purified, e.g., by Protein A or Protein G selection, sizing column chromatography), centrifugation, differential solubility, and/or by any other standard technique for the purification of proteins. Libraries of combinatorial libraries thus obtained can be screened, e.g., by incubating a heterogeneous mixture of these antibodies with a peptide derived from the HbG-Makassar variant polypeptide that has been immobilized to a surface for a period of time sufficient to allow antibody-antigen binding. Non-binding antibodies or fragments thereof can be removed by washing the surface with an appropriate buffer (e.g., a solution buffered at physiological pH (approximately 7.4) and containing physiological salt concentrations and ionic strength, and optionally containing a detergent, such as TWEEN-20®). Antibodies that remain bound can subsequently be detected, e.g., using an ELISA-based detection protocol (see, e.g., U.S. Pat. No. 4,661,445; incorporated herein by reference).

Additional techniques for screening combinatorial libraries of polypeptides (e.g., antibodies, and antigen-binding fragments thereof) for those that specifically bind to HbG-Makassar polypeptide-derived peptides include the screening of one-bead-one-compound libraries of antibody fragments. Antibody fragments can be chemically synthesized on a solid bead (e.g., using established split-and-pool solid phase peptide synthesis protocols) composed of a hydrophilic, water-swellable material such that each bead displays a single antibody fragment. Heterogeneous bead mixtures can then be incubated with an HbG-Makassar polypeptide-derived peptide that is optionally labeled with a detectable moiety (e.g., a fluorescent dye) or that is conjugated to an epitope tag (e.g., biotin, avidin, FLAG tag, HA tag) that can later be detected by treatment with a complementary tag (e.g., avidin, biotin, anti-FLAG antibody, anti-HA antibody, respectively). Beads containing antibody portions or fragments that specifically bind to an HbG-Makassar polypeptide-derived peptide can be identified by analyzing the fluorescent properties of the beads following incubation with a fluorescently-labeled antigen or complementary tag (e.g., by confocal fluorescent microscopy or by fluorescence-activated bead sorting; see, e.g., Muller et al. (J. Biol. Chem., 16500-16505, 1996); incorporated herein by reference). Beads containing antibody fragments that specifically bind to HbG-Makassar polypeptide-derived peptides can thus be separated from those that do not contain high-affinity antibody fragments. The sequence of an antibody fragment that specifically binds to an HbG-Makassar polypeptide-derived peptide can be determined by techniques known in the art, including, e.g., Edman degradation, tandem mass spectrometry, matrix-assisted laser-desorption time-of-flight mass spectrometry (MALDI-TOF MS), nuclear magnetic resonance (NMR), and 2D gel electrophoresis, among others (see, e.g., WO 2004/062553; incorporated herein by reference).

### Methods of Identifying Antibodies and Ligands

Methods for high throughput screening of antibody, antibody fragment, and ligand libraries for molecules capable of binding the HbG-Makassar polypeptide or peptide can be used to identify antibodies suitable for the uses as described herein. Such methods include in vitro display techniques known in the art, such as phage display, bacterial display, yeast display, mammalian cell display, ribosome display, mRNA display, and cDNA display, among others. The use of phage display to isolate ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules). In addition, functional antibody fragments, such as Fab and scFv fragments, have been expressed in in vitro display formats (see, for example, McCafferty et al., Nature 348:552-554, 1990; Barbas et al., Proc. Natd. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules). These techniques, among others, can be used to identify and improve the affinity of antibodies that bind to the HbG-Makassar polypeptide or peptide.

### Host Cells for Expression of Anti-HbG-Makassar Antibodies

Mammalian cells can be co-transfected with polynucleotides encoding the antibodies of some aspects and embodiments herein, which are expressed as recombinant polypeptides, and assembled into anti-HbG-Makassar antibodies by the host cell. In one embodiment, a mammalian cell is co-transfected with polynucleotides encoding the heavy and light chains of an anti-HbG-Makassar polypeptide antibody, which are expressed in the cell and assembled as the anti-HbG-Makassar antibody.

It is possible to express antibodies or antigen-binding fragments thereof in either prokaryotic or eukaryotic host cells. In certain embodiments, expression of polypeptides or antigen-binding fragments thereof is performed in eukaryotic cells, e.g., mammalian host cells, for optimal secretion of a properly folded and immunologically active antibody. Exemplary, nonlimiting mammalian host cells for expressing the recombinant antibodies or antigen-binding fragments thereof of some aspects and embodiments herein include Chinese Hamster Ovary (CHO cells) (including DHFR CHO cells, described in Urlaub and Chasin (1980, Proc. Natd. Acad. Sci. USA 77:4216-4220), used with a DHFR selectable marker, e.g., as described in Kaufman and Sharp (1982, Mol. Biol. 159:601-621), NSO myeloma cells, COS cells, HEK293T cells, SP2/0, NIH3T3, and BaF3 cells. Additional, nonlimiting cell types that may be useful for the expression of antibodies and fragments thereof include bacterial cells, such as BL-21(DE3) E. coli cells, which can be transformed with vectors containing foreign DNA according to established protocols. Additional eukaryotic cells that may be useful for expression of antibodies include yeast cells, such as auxotrophic strains of S. cerevisiae, which can be transformed and selectively grown in incomplete medium according to established procedures known in the art. When recombinant expression vectors encoding antibody genes are introduced into mammalian host cells, the antibodies are produced by culturing the host cells for a period of time sufficient to allow for expression of the antibody protein in the host cells or secretion of the antibody into the culture medium in which the host cells are grown.

Polypeptides (e.g., antibodies or antigen-binding fragments thereof) can be recovered from the culture medium using standard protein purification methods. Host cells can also be used to produce portions of intact antibodies, such as Fab fragments or scFv molecules. Also included in some aspects and embodiments herein are methods in which the above procedure is varied according to established protocols known in the art. For example, it may be desirable to transfect a host cell with DNA encoding either the light chain or the heavy chain (but not both) of an anti-HbG-Makassar antibody of some aspects and embodiments herein in order to produce an antigen-binding fragment of the antibody.

Once a HbG-Makassar-binding polypeptide (e.g., an anti-HbG-Makassar polypeptide antibody or an antigen-binding fragment thereof) of some aspects and embodiments herein has been produced by recombinant expression, it can be purified by any method known in the art, such as a method useful for purification of an immunoglobulin molecule, for example, by chromatography (e.g., ion exchange, affinity, affinity for antigen (e.g., an HbG-Makassar polypeptide or peptide) after Protein A or Protein G selection, and sizing column chromatography), centrifugation, differential solubility, or by any other standard technique for the purification of proteins. Further, an HbG-Makassar-binding polypeptide (e.g., an anti-HbG-Makassar antibody of some aspects and embodiments described herein) or an antigen-binding portion or fragment thereof, can be fused to heterologous polypeptide sequences as known in the art, for example, to facilitate purification, e.g., a histidine tag, a detectable/detectably labeled marker, and the like.

Once isolated, an anti-HbG-Makassar antibody, or antigen-binding portion or fragment thereof can, if desired, be further purified, e.g., by high performance liquid chromatography (see, e.g., Fisher, Laboratory Techniques in Biochemistry and Molecular Biology (Work and Burdon, eds., Elsevier, 1980); incorporated herein by reference), or by gel filtration chromatography, such as on a Superdex™ 75 column (Pharmacia Biotech AB, Uppsala, Sweden).

### Kits

Various aspects of this disclosure provide kits comprising a binding polypeptide or antibody, or an antigen binding portion thereof, that specifically binds to HbG-Makassar. The kit provides, in some embodiments, instructions for using the kit to detect or identify the presence of HbG-Makassar polypeptide or peptide in a sample. The instructions will generally include information about the use of the kit for binding to the cognate or target antigen, HbG-Makassar. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization. The kit can further comprise a second container comprising a suitable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of aspects and embodiments herein employs, unless otherwise indicated, techniques of molecular biology (including recombinant techniques), microbiology, cell biology, biochemistry and immunology, which are well within the knowledge and purview of the skilled artisan in the pertinent art. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of some aspects and embodiments herein, and, as such, may be considered in making and practicing some of the aspects and embodiments herein. Particularly useful techniques for particular embodiments will be discussed in the sections that follow.

The following examples are put forth so as to provide those of ordinary skill in the art with a complete disclosure and description of how to make and use the assays, screening, and therapeutic methods of some of the aspects and embodiments herein, and are not intended to limit the scope of the various aspects and embodiments herein.

### EXAMPLES

### Example 1: Generation of Anti-HbG-Makassar Variant Monoclonal Antibodies

Antibodies (monoclonal antibodies) that specifically bind to the HbG-Makassar variant polypeptide (UniProtKB Ref: P68871 (HBB_Human)), or a peptide thereof, were generated by immunizing mice with a purified HbG-Makassar peptide immunogen and employing fusion and hybridoma production methods known to those having skill in the art. The Makassar human beta-globin (HbG) mutation is E→A at amino acid position 6 of the beta-globin polypeptide. Without intending to be limiting, the protocol described below was used.

Immunization. Mice (8) were immunized with a purified (>95% purity via HPLC analysis) HbG Makassar peptide immunogen using the following protocol employing the Makassar Hb immunogen in Complete Freund's Adjuvant (CFA), Incomplete Freund's Adjuvant (IFA), or TITERMAX® gold adjuvant (TMX), (Sigma-Aldrich, St. Louis, MO), to enhance the immune response at the indicated doses. TMX gold adjuvant, an alternative to CFA, is formulated with squalene to produce a lower viscosity, stable, water-in-oil emulsion that entraps antigen, allowing for use with a variety of antigens.

| | 25 μg | 25 μg | 25 μg | 25 μg | 25 μg |
|---|---|---|---|---|---|
| # Mice, | CFA | IFA | TMX | IFA | TMX |
| Strain | Day 0 | Day 7 | Day 10 | Day 14 | Day 17 |
| 2, A/J | Makassar-Hb | HbM-A.01-KLH | | | |
| 2, C57BL/6J | | | | | |

| # Mice, | 50 μg CFA | 25 μg IFA | 25 μg IFA | 25 μg IFA |
|---|---|---|---|---|
| Strain | Day 0 | Day 10 | Day 20 | Day 30 |
| 2, A/J | Makassar-Hb | HbM-A.01-KLH | | |
| 2, C57BL/6 | | | | |

The HbG Makassar peptide immunogen (HbM-A.01-KLH in the above chart) has the following structure/sequence: amino-VHLTPAEKSAVTAC-amide, in which a carboxy-terminal cysteine (C) was added for conjugation to a carrier (i.e., keyhole limpet hemocyanin, KLH). A 28-day RIMMS (repetitive immunizations at multiple sites) protocol that involved the use of a repetitive, multiple site immunization strategy was used. RIMMS takes advantage of rapid hypermutation and affinity maturation events that occur in B cell populations localized within secondary lymphatic tissue (e.g., spleen, lymph nodes) early in response to antigenic challenges. The immunization sites used for RIMMS are typically proximal to easily accessible regional lymph nodes. The RTMMS technique allows for the somatic fusion of immune B cells undergoing germinal center maturation in draining lymph nodes, as well as in the spleen. Fusions can be performed from 7-14 days after the onset of immunization, and affinity matured murine hybridomas cell lines can be generated within a one month period. (See, e.g., E. Greenfield, 2020, Cold Spring Harbor Protocols, Cold Spring Harbor Laboratories Press; doi 10.1101/pdb.prot100313). Serum titers of each animal were assessed periodically following the immunizations.

Fusion Boost, Fusion and Screening. Selected, immunized mice were boosted with the HbG Makassar-KLH peptide (10 pg of peptide in PBS) 3 to 5 days prior to fusion. Thereafter, an animal having an appropriate serum antibody titer was sacrificed, and its spleen cells (or lymphocytes) were fused with NS1 myeloma cells in the presence of polyethylene glycol (PEG). Following this, the fused cells (hybridoma cells) were distributed (plated) into 16×96 well microtiter plates. Ten to eleven days after plating, the supernatants from hybridoma cells in the wells were screened to detect the presence of antibodies that specifically bound to HbG Makassar and showed no or no detectable binding to sickle Hb (HbS) or to wild-type (wt) beta-globin (Wt-Hb). A solid-phase ELISA was used for the screenings, in which antigens were coated on the solid substrate at 2 μg/ml. The desired binding profile of the hybridoma antibodies screened at various stages is shown below:

| | | HbM-A.01- | HbS-A.01- | Makassar-Hb | | Sickle-Hb |
|---|---|---|---|---|---|---|
| | | BSA | BSA | (HbG or HbM) | Wt-Hb | (HbS) |
| Stage | Test bleed | + | − | + | − | − |
| | Primary | + | n/a | n/a | n/a | n/a |
| | Fusion | | | | | |
| | Fusion | + | − | + | − | − |
| | Rescreen | | | | | |
| | Primary | + | n/a | n/a | n/a | n/a |
| | Subclone | | | | | |
| | Subclone | + | − | + | − | − |
| | Rescreen | | | | | |

Legend: “+” = binding, “−” = no binding, “+/−” = select both, “n/a” = assay not run during this stage

To screen for the presence of anti-HbG Makassar antibodies, the HbG Makassar peptide (amino-VHLTPAEKSAVTAC-) described above was used, as well as a purified sickle cell HbS peptide (amino-VHLTPVEKSAVTAC-amide), e.g., a recombinant peptide, and a purified wt beta (β)-globin peptide (amino-VHLTPEEKSAVTAC-amide), e.g., a recombinant peptide, as controls. The Makassar HbG peptide (referred to as HbM in this Example), the wildtype hemoglobin peptide (Wt Hb), and the sickle cell hemoglobin variant peptide (HbS) differ at amino acid position 6 of the peptide. The carboxy terminal C amino acid was added to each peptide for conjugation to a carrier, such as KLH or bovine serum albumin (BSA). In an embodiment, the recombinant HbG Makassar peptide was produced in E. coli using conventional methods.

Up to 94 antibody-secreting, positive hybridoma cell lines were expanded in 24-well plates. Supernatants from the expanded positive hybridoma cell lines were rescreened by ELISA after 3 to 5 days. Up to twenty positive hybridoma clones were frozen (one vial each with 1.5 ml supernatant sample collected at freezing) for testing prior to subcloning. Subcloning. Each selected parental hybridoma cell line was subcloned (e.g., by limiting dilution) into 1×96 well-microtiter plates and screened by ELISA 10 to 11 days after subcloning. For each parental hybridoma line, up to six positive daughter clones were expanded into 24-well plates. The supernatants from the hybridoma clones were rescreened by ELISA, and the isotypes of the antibodies produced by the expanded positive hybridoma subclones were assayed after 3 to 5 days in culture. Two positive hybridoma clones (parent lines) were selected and expanded for freezing, and their supernatants (1.5 ml) were collected at the time of expanding for freezing. Two rounds of subcloning were performed to ensure clonality of the positive hybridoma cell lines.

Selected, cloned hybridoma cell lines that produced and secreted monoclonal anti-HbG Makassar antibodies were cultured in serum-free medium to a final volume of 250 ml. Cloned hybridoma cell culture supernatants were harvested, purified over protein G resin, and dialyzed into PBS, pH 7.4 buffer. The VH and VL antibody polypeptides from selected anti-HbG Makassar antibodies secreted by the cloned hybridoma cell lines were sequenced. The sequences of representative monoclonal anti-HbG Makassar antibodies produced by the method are described supra.

### Example 2: Anti-HbG-Makassar Variant Antibody Sequencing

### Sample Preparation

Total RNA was isolated from the hybridoma cell line culture (2×10⁶ cells). RNA was treated to remove aberrant transcripts and reverse transcribed using oligo(dT) primers. Samples of the resulting cDNA were amplified in separate polymerase chain reactions (PCRs) using framework 1 and constant region primer pairs specific for either the heavy (H) or light (L) immunoglobulin chain. Reaction products were separated on an agarose gel and were size-evaluated. PCR reactions were prepared for sequencing using a PCR clean up kit and sequenced at GENEWIZ using an Illumina® NovSeq 6000.

### Sequence Analysis

DNA sequence data from all constructs were analyzed and consensus sequences for the heavy and light chains were determined. The consensus sequences were compared to all known immunoglobulin variable region sequences (e.g., Green Mountain Antibody variable region sequences) to rule out artifacts and/or process contamination. Consensus sequences were then analyzed using an online tool to verify that the sequences encoded a productive immunoglobulin molecule.

### Example 3: Anti-HbG-Makassar Antibodies Specifically Bind to the Makassar Variant Polypeptide

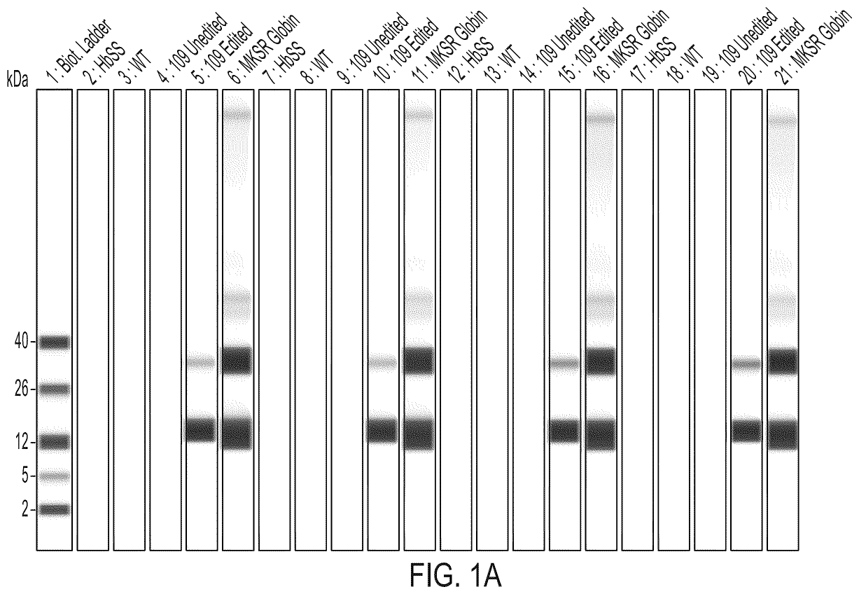

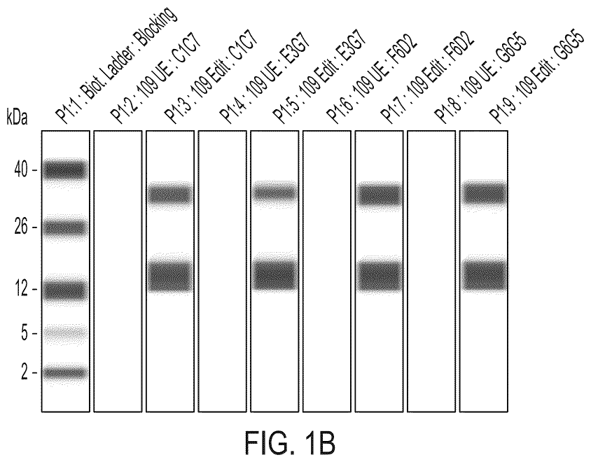

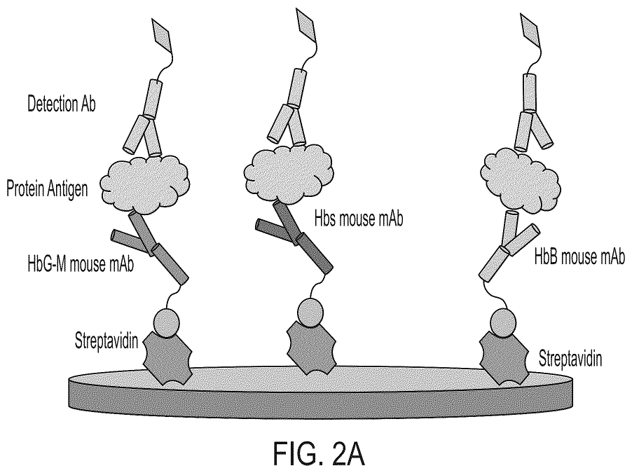

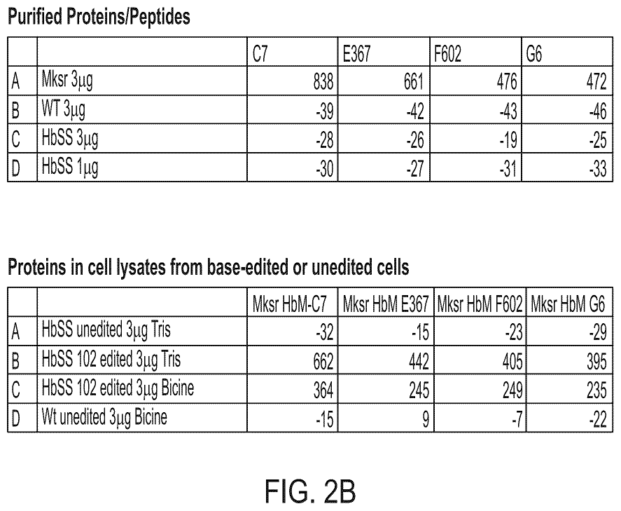

Western blot (automated immunoblot) analyses were performed to assess the binding specificity of representative monoclonal antibodies generated against the anti-HbG-Makassar variant peptide to the HbG-Makassar polypeptide as described above using an automated protein analysis system (ProteinSimple Jess protein analysis system, Bio-Techne, MN). In brief, samples (hybridoma supernatant) and reagents (e.g., target antigen, buffer) were loaded into the wells of a microtiter plate according to the manufacturer's instructions, and the automated Jess system separated the target proteins by size, added the supernatant containing antibodies, and carried out the incubation, washing, detection (e.g., chemiluminescent detection or fluorescent detection) and imaging steps, as well normalized the target antigen protein to the amount of protein added. In the analyses, monoclonal anti-HbG-Makassar antibodies produced by hybridoma clones were assayed for binding to the HbG-Makassar variant polypeptide versus wild-type hemoglobin (Hb), (β-globin) and HbS variant associated with Sickle cell disease (SCD). Antibodies produced by different hybridoma clones were assayed, including the anti-HbG-Makassar antibodies 5D6.F6.D2, 1C10.E3.G7, 5D6.G6.G4, and 5D6.G6.G5. The amino acid sequences of the VH and VL chains of anti-HbG-Makassar antibodies 1C10.E3.G7 and 5D6.G6.G5 were found to be the same. Anti-HbG-Makassar antibody 5D6.F6.D2 has the same VL chain amino acid sequence, but a different VH chain amino acid sequence compared with the VL and VH amino acid sequences of antibodies 1C10.E3.G7 and 5D6.G6.G5. As shown in FIG. 1A and FIG. 1B, representative monoclonal anti-HbG-Makassar variant polypeptide antibodies (1C10.C1.C7, 1C10.E3.G7, 5D6.G6.G5, and 5D6.F6.D2) obtained from cloned hybridoma cell lines and analyzed for binding using the automated (ProteinSimple Jess protein analysis system bound specifically to the HbG-Makassar polypeptide/peptide target antigen and showed no cross-reactivity or binding to wildtype β-globin or to sickle cell globin (HbS) associated with SCD. In particular, the monoclonal anti-HbG-Makassar antibodies 1C10.C1.C7, 1C10.E3.G7, 5D6.G6.G5, and 5D6.F6.D2 showed specific binding to HbG Makassar globin target antigen, but did not bind to wildtype β-globin or to sickle cell globin HbSS (HbS). The target antigens used in the experiments were either recombinant HbG polypeptide/peptide or HbG Makassar polypeptide obtained from lysates of base-edited HbSS cells that expressed and produced Makassar globin. Multiplex binding assays also confirmed and corroborated the specific binding of the anti-HbG Makassar monoclonal antibodies to HbG Makassar proteins, either purified, recombinant HbG Makassar protein/peptide or HbG Makassar protein/peptide present in base-edited cell lysates and lack of binding of the anti-HbG Makassar monoclonal antibodies to purified, recombinant control globin proteins/peptides, i.e., HbB or HbS, or to HbB or HbS present in cell lysates. (FIGS. 2A and 2B).

### Example 4: Three-Dimensional (3D) Models of Interactions Between the Described Anti-HbG-Makassar Antibody Proteins and an HbG Makassar Peptide

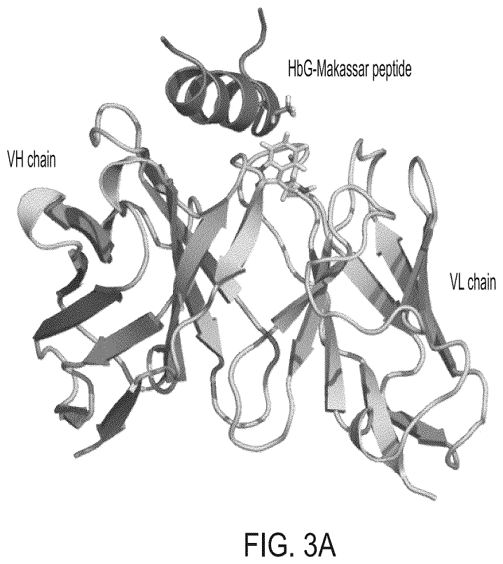

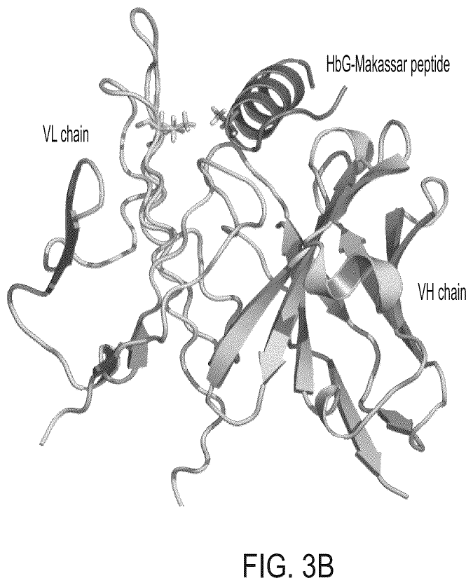

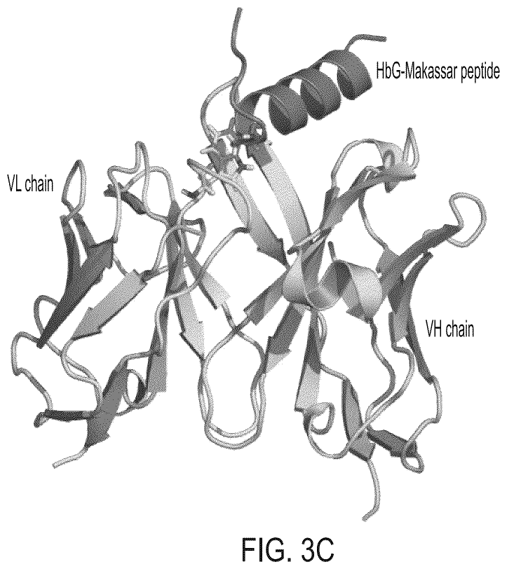

Three-dimensional (3D) protein structures were predicted and generated based on the VH and VL amino acid sequences of anti-HbG Makassar antibodies 1C10.E3.G7, 1C10.C1.C7, and 5D6.F6.D2 (Table 4 supra), and amino acids 1-19 of the HbG Makassar polypeptide using the computational neural network-based model AlphaFold. (See, e.g., J. Jumper et al., 2021, Nature, 596 (7873), 583-589; R. Evans et al., 2022, DeepMind, (doi.org/10.1101/2021.10.04.463034; A. David et al., 2022, J. Mol. Biol., 434(2): 167336). As will be appreciated by the skilled practitioner in the art, AlphaFold is an artificial intelligence (AI) program, designed as a deep learning system and developed by DeepMind, a subsidiary of Alphabet, which performs predictions of protein structure, as described in the above-listed publications. FIG. 3A shows a 3D structural model of the interaction between the VH and VL chains of the anti-HbG Makassar antibody 1C10.E3.G7 and the HbG Makassar peptide. Based on the structural model, the 1C10.E3.G7 anti-HbG Makassar antibody appears to be engaging the HbG Makassar peptide (and Makassar A6) using the VL CDR3. Makassar A6 refers to amino acid residue number 6 of HbG-Makassar, which reflects a change from glutamic acid (E) in sickle cell hemoglobin (HbS) to alanine (A) in Makassar hemoglobin (HbG) at position 6 of the Makassar protein/peptide. The closest amino acid interaction between the 1C10.E3.G7 antibody light chain (VL) and residue A6 of the HbG Makassar protein/peptide is VL amino acid residue 90, which is tryptophan (Trp), i.e., L90:Trp. FIG. 3B shows a 3D structural model of the interaction between the VH and VL chains of the anti-HbG Makassar antibody 1C10.C1.C7 and the HbG Makassar protein/peptide. Based on the structural model, the closest amino acid interaction between the 1C10.C1.C7 antibody light chain (VL) and residue A6 of the HbG Makassar protein/peptide is VL amino acid residue 16, which is leucine (Leu), i.e., L16:Leu. In this figure, the AlphaFold model quality of the antibody heavy chain is higher than that for the antibody light chain. FIG. 3C shows a 3D structural model of the interaction between the VH and VL chains of the anti-HbG Makassar antibody 5D6.F6.D2 and the HbG Makassar protein/peptide. Based on the structural model, the closest amino acid interaction between the 5D6.F6.D2 antibody light chain (VL) and residue A6 of the HbG Makassar protein/peptide is VL amino acid residue 90, which is tryptophan (Trp), i.e., L90:Trp.

### OTHER EMBODIMENTS

From the foregoing description, it will be apparent that variations and modifications may be made to some aspects and embodiments herein to adopt them to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A binding polypeptide, anti-HbG-Makassar antibody, or an antigen binding portion thereof that specifically binds to an hemoglobin G (HbG) Makassar variant polypeptide, or a peptide thereof, but fails to detectably bind or binds at reduced levels to a wild-type beta (β)-globin polypeptide and/or a sickle cell globin (HbS) polypeptide, or a peptide thereof.

2. The binding polypeptide or the antibody of claim 1, comprising one or more complementarity determining regions (CDRs) which comprise or consist of heavy chain variable region (VH) CDRs and/or light chain variable region (VL) CDRs selected from the following:
A)
VH CDR1:
GIDFSRYW;
VH CDR2:
INIDSSTI;
VH CDR3:
ARAYDGYSLDY;
VL CDR1:
SSVSY;
VL CDR2:
DTS;
VL CDR3:
RQWSSYPLT;
B)
VH CDR1:
GYTFTNYF;
VH CDR2:
INPKNGGI;
VH CDR3:
ARGSANWGAY;
VL CDR1:
QRTNC;
VL CDR2:
HDL;
VL CDR3:
QQWSSYPLT;
or
C)
VH CDR1:
GYTFTSDW;
VH CDR2:
IYPRSGST;
VH CDR3:
ARGTYYGSRSYYFDY;
VL CDR1:
SSVSY;
VL CDR2:
DTS:
VL CDR3:
RQWSSYPLT,

3. The binding polypeptide or the antibody of claim 1, which comprises or consists of:
VL CDR1
SSVSY,
VL CDR2:
DTS,
and
VL CDR3:
RQWSSYPLT
and
VH CDR1:
GIDFSRYW;
VH CDR2:
INIDSSTI;
VH CDR3:
ARAYDGYSLDY
or
VH CDR1:
GYTFTSDW;
VH CDR2:
IYPRSGST;
VH CDR3:
ARGTYYGSRSYYFDY;
VL CDR1
QRTNC;
VL CDR2:
HDL;
VL CDR3:
QQWSSYPLT
and
VH CDR1:
GYTFTNYF;
VH CDR2:
INPKNGGI;
VH CDR3:
ARGSANWGAY.
a variable heavy chain (VH) domain comprising a CDR1 comprising amino acid sequence GIDFSRYW, a CDR2 comprising amino acid sequence INIDSSTI, and a CDR3 comprising amino acid sequence ARAYDGYSLDY; or
a variable heavy chain (VH) domain comprising a CDR1 comprising amino acid sequence GYTFTSDW, a CDR2 comprising amino acid sequence IYPRSGST, and a CDR3 comprising amino acid sequence ARGTYYGSRSYYFDY; and/or
a variable light chain (VL) domain comprising a CDR1 comprising amino acid sequence SSVSY, a CDR2 comprising amino acid sequence DTS, and a CDR3 comprising amino acid sequence RQWSSYPLT.

4. The binding polypeptide or the antibody of claim 1, comprising a heavy chain variable domain (VH) sequence having at least 85%, 90%, or 95% amino acid sequence identity to the amino acid sequence:
EVQLQESGGGLVQPGGSLKLSCAASGIDFSRYWMSWVRRAPGKGL
EWIGEINIDSSTINYAPSLKDKFIISRDNAKNTLYLQMSKVRSED
TALYYCARAYDGYSLDYWGQGTSVTVSS,
and/or comprising a light chain variable domain (VL) sequence having at least 85%, 90%, or 95% amino acid sequence identity to the amino acid sequence:
QIVLTQSPAIMSASPGEKVTMTCSTSSSVSYMFWYQQKPGSSPRL
LIYDTSNLASGVPVRFSGSGSGTSYSLTISRMEAEDAATYYCRQW
SSYPLTFGAGTKLELK.

5. The binding polypeptide or the antibody of claim 1, comprising a heavy chain variable domain (VH) sequence having at least 85%, 90%, or 95% amino acid sequence identity to the amino acid sequence:
EVLLQQSGPELVKPGASVKISCKASGYTFTNYFMNWVKQSHGKSL
EWIGDINPKNGGISYNQKFKGKATLIVDKSSSTAYMELRSLTSED
SAVYYCARGSANWGAYWGQGTLVTVSA,
and/or comprising a light chain variable domain (VL) sequence having at least 85%, 90%, or 95% amino acid sequence identity to the amino acid sequence:
WWEDGYSWCSISHFQLPANQCLSHTVQRTNCSRPVSSNHVCISRG
EGHHDLQCQLKFPVRFSGSGSGTSYSLTISRMEAEDAATYYCQQW
SSYPLTFGAGTKLELK.

6. The binding polypeptide or the antibody of claim 1, comprising a heavy chain variable domain (VH) sequence having at least 85%, 90%, or 95% amino acid sequence identity to the amino acid sequence:
QVQLQQPGAELVKPGASVKMSCKASGYTFTSDWITWVKQRPGQGL
EWIGDIYPRSGSTNYNEKFKSKATLTVDISSNTAYMQLSSLTSED
SAVFYCARGTYYGSRSYYFDYWGQGTTLTVSS,
and/or comprising a light chain variable domain (VL) sequence having at least 85%, 90%, or 95% amino acid sequence identity to the amino acid sequence:
QIVLTQSPAIMSASPGEKVTMTCSTSSSVSYMFWYQQKPGSSPRL
LIYDTSNLASGVPVRFSGSGSGTSYSLTISRMEAEDAATYYCROW
SSYPLTFGAGTKLELK.

7. The binding polypeptide or the antibody of claim 1, wherein the binding polypeptide or the antibody, or a binding portion thereof, comprises an affinity tag or a detectable amino acid sequence.

8. A method of identifying an HbG-Makassar variant polypeptide or peptide, the method comprising contacting a sample with the binding polypeptide or the antibody of claim 1 for a time sufficient for the polypeptide or the antibody to bind to the HbG-Makassar variant polypeptide or peptide in the sample.

9. An isolated nucleic acid molecule that encodes the binding polypeptide or the antibody of claim 1.

10. The isolated nucleic acid molecule of claim 9, comprising a nucleic acid sequence having at least 85%, 90, 95, or 100% sequence identity to the heavy chain variable domain
(VH) nucleic acid sequence
gaggtgcagctgcaggagtctggaggtggcctggtgcagcctgga
ggatccctgaaactctcctgtgcagcctcaggaatcgattttagt
agatactggatgagttgggttcggcgggctccagggaaaggacta
gaatggattggagaaattaatatagatagcagtacaataaactat
gcaccatctctaaaggataaattcatcatctccagagacaacgcc
aaaaatacgctgtacctgcaaatgagcaaagtgagatctgaggac
acagccctttattactgtgcaagggcctatgatggttattcgttg
gactactggggtcaaggaacctcagtcaccgtctcctcag
and/or comprising a nucleic acid sequence having at least 85% sequence identity to the light chain variable domain (VL) nucleic acid sequence
caaattgttctcacccagtctccagcaatcatgtctgcatctcca
ggggagaaggtcaccatgacctgcagtaccagctcaagtgtaagt
tacatgttctggtaccagcagaagccaggatcctcccccagactc
ctgatttatgacacatccaacctggcttctggagtccctgttcgc
ttcagtggcagtgggtctgggacctcttactctctcacaatcagc
cgaatggaggctgaagatgctgccacttattactgccggcagtgg
agtagttaccccctcacgttcggtgctgggaccaagctggagctg
aaac;
gaggtcctgctgcaacaatctggacctgagctggtgaagcctggg
gcttcagtgaagatttcctgtaaggcttctggatacacgttcact
aactacttcatgaactgggtgaagcagagccatggaaagagcctt
gagtggattggagatattaatcctaagaatggtggtattagttac
aaccagaaatttaagggcaaggccacattgattgtagacaagtcc
tccagcacagcctacatggagctccgcagcctgacttctgaggac
tctgcagtctattattgtgcaagagggtcagctaactggggggct
tactggggccaagggactctggtcactgtctctgcag
and/or comprising a nucleic acid sequence having at least 85% sequence identity to the light chain variable domain (VL) nucleic acid sequence
tggtgggaagatggatacagttggtgcagcatcagccattttcag
cttcctgctaatcagtgcctcagtcatactgtccagaggacaaat
tgttctcgcccagtctccagcaatcatgtctgcatctccagggga
gaaggtcaccatgacctgcagtgccagctcaagttccctgttcgc
ttcagtggcagtgggtctgggacctcttactctctcacaatcagc
cgaatggaggctgaagatgctgccacttattactgccagcagtgg
agtagttaccccctcacgttcggtgctgggaccaagctggaactg
aaac;
or
caggtccagctgcagcagcctggggctgagcttgtgaagcctggg
gcttcagtgaagatgtcctgcaaggcttctggctacaccttcacc
agcgactggataacctgggtgaagcagaggcctggacaaggcctt
gagtggattggagatatttatcctcgtagtggtagtactaactac
aatgagaagttcaagagcaaggccacactgactgtagatatatcc
tccaacacagcctacatgcagctcagcagcctgacatctgaggac
tctgcggtcttttactgtgcaagagggacttactacggtagtagg
tcctactactttgactactggggccaaggcaccactctcacagtc
tcctcag
and/or comprising a nucleic acid sequence having at least 85% sequence identity to the light chain variable domain (VL) nucleic acid sequence
caaattgttctcacccagtctccagcaatcatgtctgcatctcca
ggggagaaggtcaccatgacctgcagtaccagctcaagtgtaagt
tacatgttctggtaccagcagaagccaggatcctcccccagactc
ctgatttatgacacatccaacctggcttctggagtccctgttcgc
ttcagtggcagtgggtctgggacctcttactctctcacaatcagc
cgaatggaggctgaagatgctgccacttattactgccggcagtgg
agtagttaccccctcacgttcggtgctgggaccaagctggagctg
aaac.

11. A method of identifying and/or selecting a subject expressing an HbG-Makassar polypeptide, the method comprising:
(a) contacting a sample obtained from the subject with the binding polypeptide, the antibody, or an antigen binding portion thereof, of claim 1;
(b) detecting specific binding between the binding polypeptide, the antibody, or the antigen binding portion thereof, and an HbG-Makassar polypeptide in the sample; and
(c) identifying and/or selecting the subject as expressing an HbG-Makassar polypeptide based on the detecting step (b).

12. A method of monitoring a subject for the production of HbG-Makassar polypeptide, the method comprising:
(a) contacting a sample obtained from the subject with the binding polypeptide, the antibody, or an antigen binding portion thereof, of claim 1 at a first time point and detecting specific binding between the binding polypeptide, the antibody, or the antigen binding portion thereof, and an HbG-Makassar polypeptide in the sample;
(b) contacting a sample obtained from the subject with the binding polypeptide, the antibody, or the antigen binding portion thereof, of claim 1 at one or more additional time points and detecting specific binding between the binding polypeptide or the antibody and an HbG-Makassar polypeptide in the sample; and
(c) monitoring that the subject is expressing the HbG-Makassar polypeptide by detecting the same level or a greater level of the HbG-Makassar polypeptide in the subject's sample in step (b) versus step (a).

13. A method of assessing a relative or absolute level of HbG-Makassar hemoglobin in a subject expressing an HbG-Makassar polypeptide, the method comprising:
(a) contacting a sample obtained from the subject with the binding polypeptide, the antibody, or an antigen binding portion thereof, of claim 1;
(b) detecting specific binding between the binding polypeptide, the antibody, or the antigen binding portion thereof, and an HbG-Makassar polypeptide in the sample; and
(c) assessing a relative or absolute level of at least 30% of HbG-Makassar hemoglobin in the sample based on the detecting step (b); wherein said level of HbG-Makassar in the subject is sufficient to prevent sickling by HbG-S hemoglobin in the subject.

14. The method of claim 13, wherein the anti-HbG-Makassar antibody comprises 1C10.E3.G7, 1C10.C1.C7, or 5D6.F6.D2, or an antigen binding portion thereof.

15. A composition comprising the binding polypeptide or the antibody of claim 1, or an antigen binding fragment thereof, or the nucleic acid encoding the binding polypeptide or the antibody.

16. A vector comprising a nucleic acid molecule that encodes the binding polypeptide or the antibody of claim 1.

17. A cell comprising the vector of claim 15.

18. A kit comprising the binding polypeptide, the antibody, or an antigen binding portion thereof, of claim 1 or the nucleic acid molecule encoding the binding polypeptide or the antibody.

19. The binding polypeptide, the anti-HbG-Makassar antibody, or an antigen binding portion thereof, of claim 1, wherein the polypeptide, antibody, or the antigen binding portion thereof specifically binds to a hemoglobin G (HbG) Makassar peptide comprising the amino acid sequence VHLTPAEKSAVTA.

20. The binding polypeptide, the anti-HbG-Makassar antibody, or an antigen binding portion thereof, of claim 18, wherein the polypeptide, antibody, or the antigen binding portion thereof specifically binds to a hemoglobin G (HbG) Makassar peptide comprising the amino acid sequence VHLTPAEKSAVTA, but fails to detectably bind or binds at reduced levels to a sickle cell HbS peptide comprising the amino acid sequence VHLTPVEKSAVTA and/or to a wildtype beta-globin peptide comprising the amino acid sequence VHLTPEEKSAVTA.


SEQUENCE LISTING (60 sequences)

SEQ ID NO: 1 - protein, 8 aa, synthetic construct
GIDFSRYW

SEQ ID NO: 2 - protein, 8 aa, synthetic construct
INIDSSTI

SEQ ID NO: 3 - protein, 11 aa, synthetic construct
ARAYDGYSLDY

SEQ ID NO: 4 - protein, 5 aa, synthetic construct
SSVSY

SEQ ID NO: 5 - skipped

SEQ ID NO: 6 - protein, 9 aa, synthetic construct
RQWSSYPLT

SEQ ID NO: 7 - protein, 8 aa, synthetic construct
GYTFTNYF

SEQ ID NO: 8 - protein, 8 aa, synthetic construct
INPKNGGI

SEQ ID NO: 9 - protein, 10 aa, synthetic construct
ARGSANWGAY

SEQ ID NO: 10 - protein, 5 aa, synthetic construct
QRTNC

SEQ ID NO: 11 - skipped

SEQ ID NO: 12 - protein, 9 aa, synthetic construct
QQWSSYPLT

SEQ ID NO: 13 - protein, 8 aa, synthetic construct
GYTFTSDW

SEQ ID NO: 14 - protein, 8 aa, synthetic construct
IYPRSGST

SEQ ID NO: 15 - protein, 15 aa, synthetic construct
ARGTYYGSRSYYFDY

SEQ ID NO: 16 - protein, 118 aa, synthetic construct
EVQLQESGGGLVQPGGSLKLSCAASGIDFSRYWMSWVRRAPGKGLEWIGEINIDSSTINYAPSLKDKFIISRDNAKNTLYLQMSKVRSEDTALYYCARAYDGYSLDYWGQGTSVTVSS

SEQ ID NO: 17 - protein, 106 aa, synthetic construct
QIVLTQSPAIMSASPGEKVTMTCSTSSSVSYMFWYQQKPGSSPRLLIYDTSNLASGVPVRFSGSGSGTSYSLTISRMEAEDAATYYCRQWSSYPLTFGAGTKLELK

SEQ ID NO: 18 - protein, 117 aa, synthetic construct
EVLLQQSGPELVKPGASVKISCKASGYTFTNYFMNWVKQSHGKSLEWIGDINPKNGGISYNQKFKGKATLIVDKSSSTAYMELRSLTSEDSAVYYCARGSANWGAYWGQGTLVTVSA

SEQ ID NO: 19 - protein, 106 aa, synthetic construct
WWEDGYSWCSISHFQLPANQCLSHTVQRTNCSRPVSSNHVCISRGEGHHDLQCQLKFPVRFSGSGSGTSYSLTISRMEAEDAATYYCQQWSSYPLTFGAGTKLELK

SEQ ID NO: 20 - protein, 122 aa, synthetic construct
QVQLQQPGAELVKPGASVKMSCKASGYTFTSDWITWVKQRPGQGLEWIGDIYPRSGSTNYNEKFKSKATLTVDISSNTAYMQLSSLTSEDSAVFYCARGTYYGSRSYYFDYWGQGTTLTVSS

SEQ ID NO: 21 - protein, 13 aa, synthetic construct
VHLTPAEKSAVTA

SEQ ID NO: 22 - protein, 13 aa, synthetic construct
VHLTPVEKSAVTA

SEQ ID NO: 23 - protein, 13 aa, synthetic construct
VHLTPEEKSAVTA

SEQ ID NO: 24 - DNA, 355 bp, synthetic construct
  source 1..355: mol_type = other DNA
gaggtgcagctgcaggagtctggaggtggcctggtgcagcctggaggatccctgaaactctcctgtgcagcctcaggaatcgattttagtagatactggatgagttgggttcggcgggctccagggaaaggactagaatggattggagaaattaatatagatagcagtacaataaactatgcaccatctctaaaggataaattcatcatctccagagacaacgccaaaaatacgctgtacctgcaaatgagcaaagtgagatctgaggacacagccctttattactgtgcaagggcctatgatggttattcgttggactactggggtcaaggaacctcagtcaccgtctcctcag

SEQ ID NO: 25 - DNA, 319 bp, synthetic construct
  source 1..319: mol_type = other DNA
caaattgttctcacccagtctccagcaatcatgtctgcatctccaggggagaaggtcaccatgacctgcagtaccagctcaagtgtaagttacatgttctggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccggcagtggagtagttaccccctcacgttcggtgctgggaccaagctggagctgaaac

SEQ ID NO: 26 - DNA, 352 bp, synthetic construct
  source 1..352: mol_type = other DNA
gaggtcctgctgcaacaatctggacctgagctggtgaagcctggggcttcagtgaagatttcctgtaaggcttctggatacacgttcactaactacttcatgaactgggtgaagcagagccatggaaagagccttgagtggattggagatattaatcctaagaatggtggtattagttacaaccagaaatttaagggcaaggccacattgattgtagacaagtcctccagcacagcctacatggagctccgcagcctgacttctgaggactctgcagtctattattgtgcaagagggtcagctaactggggggcttactggggccaagggactctggtcactgtctctgcag

SEQ ID NO: 27 - DNA, 319 bp, synthetic construct
  source 1..319: mol_type = other DNA
tggtgggaagatggatacagttggtgcagcatcagccattttcagcttcctgctaatcagtgcctcagtcatactgtccagaggacaaattgttctcgcccagtctccagcaatcatgtctgcatctccaggggagaaggtcaccatgacctgcagtgccagctcaagttccctgttcgcttcagtggcagtgggtctgggacctcttactctctcacaatcagccgaatggaggctgaagatgctgccacttattactgccagcagtggagtagttaccccctcacgttcggtgctgggaccaagctggaactgaaac

SEQ ID NO: 28 - DNA, 367 bp, synthetic construct
  source 1..367: mol_type = other DNA
caggtccagctgcagcagcctggggctgagcttgtgaagcctggggcttcagtgaagatgtcctgcaaggcttctggctacaccttcaccagcgactggataacctgggtgaagcagaggcctggacaaggccttgagtggattggagatatttatcctcgtagtggtagtactaactacaatgagaagttcaagagcaaggccacactgactgtagatatatcctccaacacagcctacatgcagctcagcagcctgacatctgaggactctgcggtcttttactgtgcaagagggacttactacggtagtaggtcctactactttgactactggggccaaggcaccactctcacagtctcctcag

SEQ ID NO: 29 - protein, 13 aa, synthetic construct
SRLEEELRRRLTE

SEQ ID NO: 30 - protein, 14 aa, synthetic construct
GKPIPNPLLGLDST

SEQ ID NO: 31 - protein, 10 aa, synthetic construct
EQKLISEEDL

SEQ ID NO: 32 - protein, 9 aa, synthetic construct
YPYDVPDYA

SEQ ID NO: 33 - protein, 12 aa, synthetic construct
PDRVRAVSHWSS

SEQ ID NO: 34 - protein, 11 aa, synthetic construct
MASMTGGQQMG

SEQ ID NO: 35 - protein, 18 aa, synthetic construct
TKENPRSNQEESYDDNES

SEQ ID NO: 36 - protein, 6 aa, synthetic construct
CCPGCC

SEQ ID NO: 37 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 38 - DNA, 628 bp, Homo sapiens
  source 1..628: mol_type = genomic DNA
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 39 - protein, 25 aa, synthetic construct
EVQLQESGGGLVQPGGSLKLSCAAS

SEQ ID NO: 40 - protein, 17 aa, synthetic construct
MSWVRRAPGKGLEWIGE

SEQ ID NO: 41 - protein, 38 aa, synthetic construct
NYAPSLKDKFIISRDNAKNTLYLQMSKVRSEDTALYYC

SEQ ID NO: 42 - protein, 11 aa, synthetic construct
WGQGTSVTVSS

SEQ ID NO: 43 - protein, 26 aa, synthetic construct
QIVLTQSPAIMSASPGEKVTMTCSTS

SEQ ID NO: 44 - protein, 17 aa, synthetic construct
MFWYQQKPGSSPRLLIY

SEQ ID NO: 45 - protein, 36 aa, synthetic construct
NLASGVPVRFSGSGSGTSYSLTISRMEAEDAATYYC

SEQ ID NO: 46 - protein, 10 aa, synthetic construct
FGAGTKLELK

SEQ ID NO: 47 - protein, 25 aa, synthetic construct
EVLLQQSGPELVKPGASVKISCKAS

SEQ ID NO: 48 - protein, 17 aa, synthetic construct
MNWVKQSHGKSLEWIGD

SEQ ID NO: 49 - protein, 38 aa, synthetic construct
SYNQKFKGKATLIVDKSSSTAYMELRSLTSEDSAVYYC

SEQ ID NO: 50 - protein, 11 aa, synthetic construct
WGQGTLVTVSA

SEQ ID NO: 51 - protein, 26 aa, synthetic construct
WWEDGYSWCSISHFQLPANQCLSHTV

SEQ ID NO: 52 - protein, 17 aa, synthetic construct
SRPVSSNHVCISRGEGH

SEQ ID NO: 53 - protein, 36 aa, synthetic construct
QCQLKFPVRFSGSGSGTSYSLTISRMEAEDAATYYC

SEQ ID NO: 54 - protein, 25 aa, synthetic construct [sequence omitted]

SEQ ID NO: 55 - protein, 17 aa, synthetic construct
ITWVKQRPGQGLEWIGD

SEQ ID NO: 56 - protein, 38 aa, synthetic construct
NYNEKFKSKATLTVDISSNTAYMQLSSLTSEDSAVFYC

SEQ ID NO: 57 - protein, 11 aa, synthetic construct
WGQGTTLTVSS

SEQ ID NO: 58 - protein, 14 aa, synthetic construct
  MOD_RES 14: note = AMIDATION; note = Cysteine amide
VHLTPAEKSAVTAC

SEQ ID NO: 59 - protein, 14 aa, synthetic construct
  MOD_RES 14: note = AMIDATION; note = Cysteine amide
VHLTPVEKSAVTAC

SEQ ID NO: 60 - protein, 14 aa, synthetic construct
  MOD_RES 14: note = AMIDATION; note = Cysteine amide
VHLTPEEKSAVTAC